(19) United States
(12) Patent Application Publication
(10) Pub. No.: US 2010/0143503 A1
(43) Pub. Date: Jun. 10, 2010

(54) COMBINATIONS OF NITRIC OXIDE AND SULFIDE AND METHODS OF USE AND MANUFACTURE THEREOF

(75) Inventors: Csaba Szabo, Seattle, WA (US); Kevin James Tomaselli, San Diego, CA (US)

Correspondence Address: LEE & HAYES, PLLC, 601 W. RIVERSIDE AVENUE, SUITE 1400, SPOKANE, WA, 99201, US

(73) Assignee: Ikaria, Inc., Seattle, WA (US)

(21) Appl. No.: 12/520,772

(22) PCT Filed: Dec. 20, 2007
(86) PCT No.: PCT/US07/88402

§ 371 Date: Feb. 12, 2010

Related U.S. Application Data
(60) Provisional application No. 60/877,051, filed on Dec. 22, 2006.
(60) Provisional application No. 60/896,739, filed on Mar. 23, 2007.

(51) Int. Cl. A61K 33/04; A61K 33/00; C12N 5/02; A61M 16/12; A61M 37/00

(52) U.S. Cl. 424/706; 424/718; 435/375; 128/203.12; 604/23

(57) ABSTRACT

The present invention provides methods of reducing the cytotoxic effects of nitric oxide and sulfides comprising coadministering nitric oxide with sulfide. In addition, the present invention provides novel pharmaceutical compositions comprising both nitric oxide and sulfide. The methods and compositions of the present invention may be used in the treatment or prevention of a variety of diseases and disorders, and also in the prevention of cell or tissue damage, including that resulting from ischemi hypoxia.

### CROSS-REFERENCES TO RELATED APPLICATIONS

This application claims the benefit under 35 U.S.C. §119(e) of U.S. Provisional Patent Application No. 60/877,051 filed Dec. 22, 2006; and U.S. Provisional Patent Application No. 60/896,739 filed Mar. 23, 2007; both of these provisional applications are incorporated herein by reference in their entireties.

### BACKGROUND OF THE INVENTION

1. Field of the Invention

The present invention relates to the treatment and prevention of cell damage and inflammation using sulfide compositions. In addition, the present invention relates to combinations of nitric oxide and sulfide, including the co-administration of pharmaceutical compositions comprising nitric oxide with pharmaceutical compositions comprising sulfide, as well as stable pharmaceutical compositions comprising both nitric oxide and sulfide. The invention further relates to the use of such compositions to treat and protect cells and animals from injury, disease, and premature death.

2. Description of the Related Art

Recently, the number of identified gaseous autocrine/paracrine messengers has expanded to include nitric oxide (NO), hydrogen sulfide (H₂S), carbon monoxide (CO)(Leffler, et al., Journal of Applied Physiology (2006) 100:1065-1076). These gaseous mediators are synthesized in the body and are both regulatory and physiological mediators.

The action of nitric oxide (NO) is considered regulatory in maintaining normal physiological homeostasis in humans and animals, i.e., host-defense, vascular tone, neurotransmission, bronchodilatation and inhibition of platelet function (see Giustarini et al., Clinica Chimica Acta (2003) 330:85-98). NO mediates blood pressure, learning and memory, immune responses, and inflammatory responses (see Thippeswamy et al., Histol. Histopathol. (2006) 21:445-458). In addition, the actions of NO have been observed in pathological conditions such as arthritis, arteriosclerosis, cancer, diabetes, some neurodegenerative diseases and stroke (see Giustarini et al., Clinica Chimica Acta (2003) 330:85-98).

NO gas is approved by the U.S. Food and Drug Administration (FDA) for use in the treatment of neonatal respiratory distress and may be useful in treating other human and animal diseases or injuries, including myocardial infarction, stroke, hemorrhage, and major surgery. NO was shown to be effective in newborn children experiencing respiratory distress in part because it causes vasodilatation of the lung vasculature (see Kinsella et al., Lancet (1992) 340:818-820; Rich et al., Anesthesiology (1993) 78:413-416). Furthermore, nitric oxide was shown to have pharmaceutical action in animals and humans (see U.S. Pat. No. 5,485,827).

In biological systems, NO can react with various molecules, including but not limited to molecular oxygen, superoxide anion (O₂⁻), or transition metals, yielding reactive nitrogen oxide species (RNOS) and metal-nitrosyl adducts (see Giustarini et al., Clinica Chimica Acta (2003) 330:85-98). NO reactivity may result in harmful effects that may be due to the direct actions of NO, or to molecules resulting from the metabolism or chemical transformation of NO to peroxynitrite (ONOO—), a reactive cytotoxic oxidant species and potent cytotoxin. Peroxynitrite (ONOO—) is formed from the rapid interaction of nitric oxide (NO) and superoxide (O₂⁻). The half-life of peroxynitrite is short (˜1 second), but sufficient to allow significant interactions with most biomolecules. When peroxynitrite is produced by NO, it may lead to cellular damage that may produce numerous pathophysiologic conditions such as localized inflammation, ischemia-reperfusion injury and shock (see Liaudet et al., Crit Care Med. (2000) 28:N37).

It was recently demonstrated that H₂S (hydrogen sulfide) gas, a potent inhibitor of oxygen consumption, can reduce metabolism and protect mice and rats from hypoxic injuries. It was shown that treatment with sulfur and other chalcogenides induces stasis of biological matter and protects biological matter from hypoxic and ischemic injury (PCT Publication No. WO2005/041655). Although hydrogen sulfide gas has not been typically considered a medical gas, this unexpected result presents exciting possibilities for the treatment or prevention of a number of animal and human diseases, particularly hypoxia and ischemia-related diseases and injuries.

Sulfide has many physiological actions in mammals, including, but not limited to, vasodilatation, cytoprotection, metabolic depression (or stasis), and anti-inflammation. Sulfide has not yet been approved by the FDA for use in invasive medical intervention. However, when administered either parenterally or by inhalation/ventilation to mammals, sulfide reduces injury and enhances survivability in myocardial infarction, cardiac surgery, lethal hemorrhage, cerebral and hepatic ischemia, and lethal hypoxia. Sulfide may reduce injury or enhance survivability in similar or other human diseases or injuries.

Inhalation or exposure to low doses of sulfide gas may produce eye irritation, cough, or nasal symptoms. Inhalation of high doses of sulfide may produce respiratory distress, (shortness of breath), headache, nausea, cardiovascular symptoms due to hypotension or loss of consciousness.

The pharmaceutical and medical uses of nitric oxide or sulfide may be limited by certain undesirable side-effects. Thus, there is clearly a need in the art for improved nitric oxide and sulfide compositions and methods of use thereof, which have reduced cytotoxicity or other undesired side-effects, as compared to currently available nitric oxide and sulfide formulations.

### BRIEF SUMMARY OF THE INVENTION

The present invention provides methods and compositions that reduce the toxic effects of nitric oxide and sulfide and may, therefore be used in the treatment and prevention of disease, disorders, and conditions that benefit from treatment with nitric oxide or sulfide. These methods and compositions may be utilized for a variety of purposes and may be administered to various biological matter, including cells, tissues, organs, organisms, and animals, including humans and other mammals.

In one embodiment, the present invention provides a method of reducing a cytotoxic effect of nitric oxide in a biological matter, comprising administering to the biological matter nitric oxide in combination with sulfide.

In another embodiment, the present invention provides a method of reducing a cytotoxic effect of sulfide in a biological matter, comprising administering to the biological matter sulfide in combination with nitric oxide.

In certain embodiments of methods of the present invention, nitric oxide and sulfide are administered as gases. In other embodiments, nitric oxide and sulfide are administered as liquids. In one embodiment, nitric oxide is administered as a gas and sulfide is administered as a liquid. In another embodiment, nitric oxide is administered as a liquid and sulfide is administered as a gas. In particular embodiments, nitric oxide and sulfide are administered concurrently. In one embodiment, sulfide is administered prior to administration of nitric oxide. In one embodiment, nitric oxide is administered prior to administration of said sulfide.

In one related embodiment, the present invention includes a method of treating or preventing a disease, disorder, or condition that benefits from treatment with nitric oxide comprising administering to a patient an effective amount of nitric oxide in combination with sulfide. In one embodiment, a therapeutically effective amount of nitric oxide is administered in combination with an amount of sulfide sufficient to reduce cytotoxicity or another undesirable side-effect associated with nitric oxide. In particular embodiments, the disease, disorder or condition is a respiratory, cardiovascular, pulmonary, or blood disease or disorder, a tumor, an infection, inflammation, shock, sepsis, or stroke, in a patient,

In a further embodiment, the present invention provides a method of preventing or reducing injury to, or enhancing survivability of, a biological material exposed to ischemic or hypoxic conditions, comprising contacting the biological material with an effective amount of sulfide in combination with nitric oxide. In one embodiment, the biological material is contacted with a therapeutically effective amount of sulfide in combination with an amount of nitric oxide sufficient to reduce cytotoxicity or an undesirable side-effect associated with sulfide. In one embodiment, the biological material is contacted with the sulfide and nitric oxide before being exposed to the ischemic or hypoxic conditions. In another embodiment, the biological material is contacted with the sulfide and nitric oxide during exposure to the ischemic or hypoxic conditions. In yet another embodiment, the biological material is contacted with the sulfide and nitric oxide after being exposed to the ischemic or hypoxic conditions.

In particular embodiments of methods of the present invention, the ischemic or hypoxic conditions result from an injury to the biological material, the onset or progression of a disease that adversely affects the biological material, or hemorrhaging of the biological material. In certain embodiments, the biological material is contacted with sulfide and nitric oxide before the injury, before the onset or progression of the disease, or before hemorrhaging of the biological material. In one embodiment, the injury is from an external physical source.

In certain embodiment of methods of the present invention, the biological material is to be transplanted. In others, the biological material is at risk for reperfusion injury or hemorrhagic shock.

In particular embodiments of the present invention, a combination of nitric oxide and sulfide is administered at a therapeutically effective amount. In certain instances, the amount of either or both nitric oxide and sulfide present in a therapeutically effective amount of a combination is less than the amount of nitric oxide of sulfide that is therapeutically effective when administered alone. In other embodiments, the amount of either or both nitric oxide and sulfide is administered in an amount that is greater than the amount of nitric oxide or sulfide that may be safely administered alone.

In another embodiment, the present invention provides a gaseous pharmaceutical composition comprising nitric oxide and sulfide.

In another related embodiment, the present invention provides a liquid pharmaceutical composition comprising sulfide and nitric oxide.

The present invention further provides systems and devices for the preparation and administration of gas and liquid compositions comprising nitric oxide and sulfide. In one embodiment, the present invention includes a device for the metered coadministration of nitric oxide and sulfide to a patient, comprising a first compartment containing nitric oxide gas, a second compartment containing sulfide gas, wherein said first and second compartments are attached to a device for mixing the contained nitric oxide and sulfide gas prior to administration to a patient.

In another embodiment, the present invention includes a device for the metered coadministration of nitric oxide and sulfide to a patient, characterized by a gas feed system including a first line feeding nitric oxide, a second line feeding sulfide, a shut-off valve in the first line, a shut-off valve in the second line, wherein the first and second lines are in flow communication with a third line, whereby upon opening both shut-off valves to open flow nitric oxide and sulfide may flow through the first and second lines and into the third line, where they are mixed, and a device for delivering the resulting mixture of nitric oxide and sulfide to the patient, wherein said device is in flow communication with the third line. In particular embodiments, the device further comprises a fourth line feeding air and a shut-off valve in the fourth line, wherein the fourth line is in flow communication with the third line, whereby upon opening all shut-off valves to open flow nitric oxide, sulfide, and air may flow through the first, second, and third lines and into the third line, where they are mixed.

In various embodiments of methods of the present invention, the nitric oxide gas and hydrogen sulfide gas are administrated to a patient or other biological matter, or biological is contacted by inhalation, e.g., through the use of a nebulizer, injection, catheterization, immersion, lavage, perfusion, topical application, absorption, adsorption, or oral administration.

In particular embodiments of methods of the present invention, administering or contacting is performed intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, or via a lavage.

In one particular embodiment, the present invention provides a method for treating or preventing a cardiovascular disease or disorder in a patient in need thereof comprising administering a therapeutically effective amount of a gas or liquid composition comprising nitric oxide and sulfide to a patient. In certain embodiments, the cardiovascular disease is myocardial or heart failure.

In another embodiment, the present invention includes a method for treating or preventing inflammatory disease or disorder in a patient in need thereof administration of a gas or liquid composition comprising nitric oxide and sulfide the composition to a patient.

In a further related embodiment, the present invention provides a method for treating or preventing a blood disorder in a patient in need thereof comprising administering a therapeutically effective amount of a gas or liquid composition comprising nitric oxide and sulfide to a patient. In one embodiment, the blood disorder is sickle cell disease.

The present invention also provides methods of preparing or manufacturing gas and liquid compositions comprising nitric oxide and sulfide.

In one embodiment, the present invention includes a method of preparing a composition comprising nitric oxide and sulfide suitable for administration to an animal, comprising: dissolving one equivalent of hydrogen sulfide gas into one equivalent of liquid, thereby producing a composition of sulfide; dissolving nitric oxide gas into the resulting composition; and adjusting the pH to a pH in the range of 6.5 to 8.5, thereby producing a liquid composition of nitric oxide and sulfide suitable for administration to an animal. In one embodiment, the liquid is sodium hydroxide.

In another embodiment, the present invention includes a method of preparing a liquid composition of nitric oxide and sulfide suitable for administration to an animal, comprising: dissolving sodium sulfide nonahydrate into liquid, thereby producing a saturated composition of sulfide; and dissolving nitric oxide gas into the resulting composition; and adjusting the pH of the composition to a pH in the range of 6.5 to 8.5, thereby producing a liquid composition of nitric oxide and sulfide suitable for administration to an animal. In one embodiment, said liquid is oxygen-free, deionized water.

In a further embodiment, the present invention includes a method of preparing a composition of nitric oxide and sulfide suitable for administration to an animal, comprising: saturating nitric oxide gas in a liquid; adding sodium sulfide into the composition; and adjusting the pH of the resulting composition to a pH in the range of 6.5 to 8.5, thereby producing a liquid composition of nitric oxide and sulfide suitable for administration to an animal. In one embodiment, the liquid is phosphate buffer. In particular embodiment, the sodium sulfide is dissolved in an excess of liquid, thereby producing a saturated composition of sulfide. In one embodiment, the resulting composition comprises nitric oxide at a concentration in the range of 0.1 mM to 1.9 mM. In another embodiment, the phosphate buffer has a concentration in the range of 0.1 mM to 1 mM. In one embodiment, the liquid has a pH in the range of 7.3 to 7.6. In one embodiment, the nitric oxide gas is 20° C.

In yet another related embodiment, the present invention provides a method of preparing a composition of nitric oxide and sulfide suitable for administration to an animal, comprising: saturating nitric oxide gas in a liquid; dissolving hydrogen sulfide gas into the composition; and adjusting the pH of the resulting composition to a pH in the range of 6.5 to 8.5, thereby producing a liquid composition of nitric oxide and sulfide suitable for administration to an animal. In particular embodiments, said liquid is phosphate buffer. In certain embodiments, the composition comprising nitric oxide at a concentration in the range of 0.1 mM to 1.9 mM. In one embodiment, said phosphate buffer has a concentration in the range of 0.1 mM to 1 mM. In one embodiment, said liquid has a pH in the range of 7.3 to 7.6. In one embodiment, the temperature of said nitric oxide gas is 20° C.

In various embodiments of the methods of the present invention for of preparing compositions comprising nitric oxide and sulfide, the nitric oxide gas in the resulting liquid composition is in the range of 10 ppm to 80 ppm. In one embodiment, the pH is adjusted by the addition of one or more of hydrogen chloride, carbon dioxide, sodium hydroxide, and hydrogen sulfide. In another embodiment, the pH is adjusted by dissolving nitric oxide, and/or hydrogen sulfide into the composition. In certain embodiments, the osmolarity of the composition is adjusted to an osmolarity in the range of 250-350 mOsmol/L. Particular embodiments further comprise dispensing the composition under inert atmosphere or noble gas into light-protective vials. Other embodiments further comprise adding an excipient to the composition. In particular embodiments, the excipient is selected from the group consisting of: chelating agents, pH modifying agents, reducing agents, free radical scavengers, and preservatives. In one embodiment, the oxygen content in the resulting composition is less than or equal to 5 μM for about six months.

BRIEF DESCRIPTION OF THE SEVERAL VIEWS OF THE DRAWING(S)

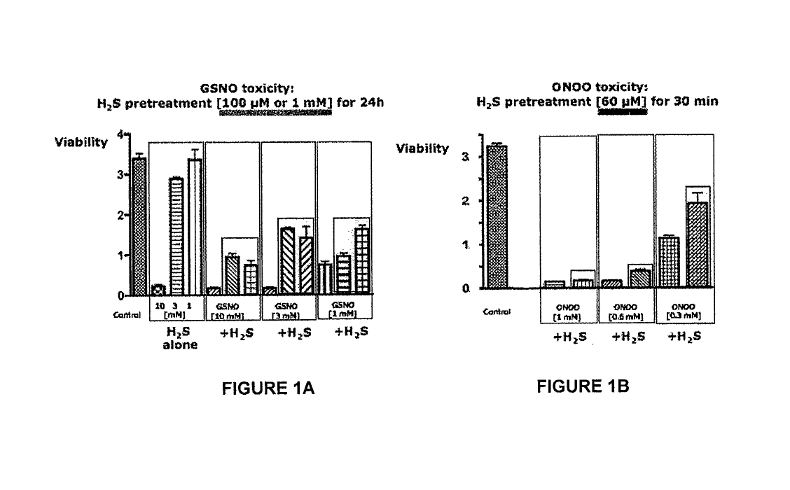

FIGS. 1A and 1B are graphs depicting the protective effects of liquid sulfide pretreatment prior to treatment with the indicated concentrations of either S-nitrosoglutathione (GSNO) or peroxynitrite (ONOO—). The graph provides cell viability measured three hours after GSNO or ONOO— treatment (n=4-6). As shown, liquid sulfide pretreatment reduced the cytotoxic effects of both GSNO and ONOO— in a concentration dependent manner.

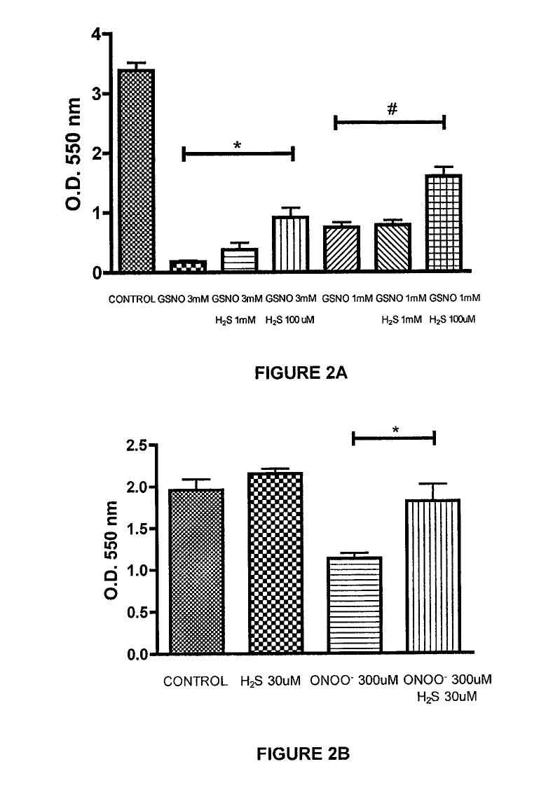

FIG. 2 demonstrates the cytoprotective effects of liquid sulfide pretreatment of macrophages for 30 minutes. FIG. 2A is a graph showing the viability of murine J774 macrophages following treatment with GSNO or ONOO— at the indicated concentrations, in the absence or presence of H₂S pretreatment for 30 minutes at the indicated concentrations. FIG. 2B is a graph showing the viability of murine J774 macrophages following treatment for 30 minutes with H₂S alone, ONOO— alone, or the combination of H₂S and ONOO—.

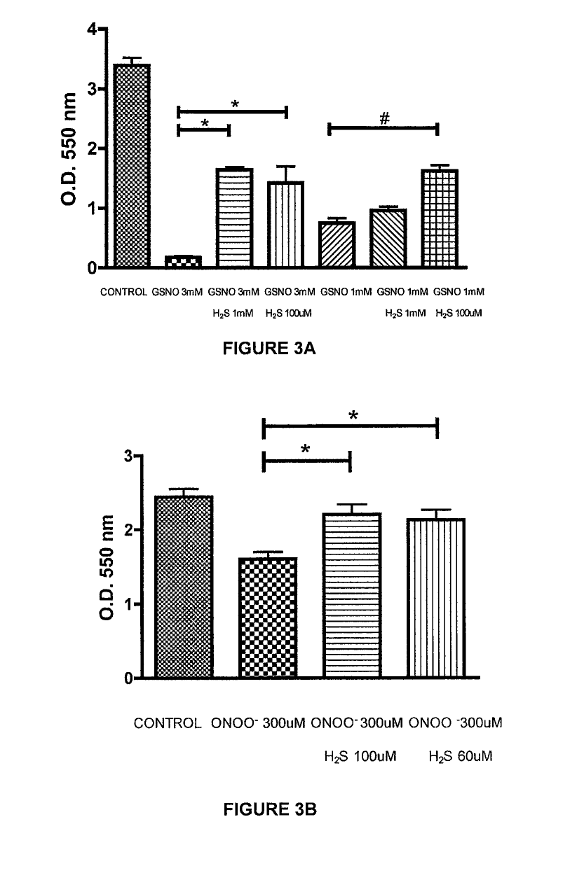

FIG. 3 demonstrates the cytoprotective effects of liquid sulfide pretreatment of macrophages for 24 hours. FIG. 3A is a graph showing the viability of murine J774 macrophages following treatment with GSNO or ONOO— at the indicated concentrations, in the absence or presence of H₂S pretreatment for 24 hours at the indicated concentrations. FIG. 3B is a graph showing the viability of murine J774 macrophages following treatment for 24 hours with H₂S alone, ONOO— alone, or the combination of H₂S and ONOO—.

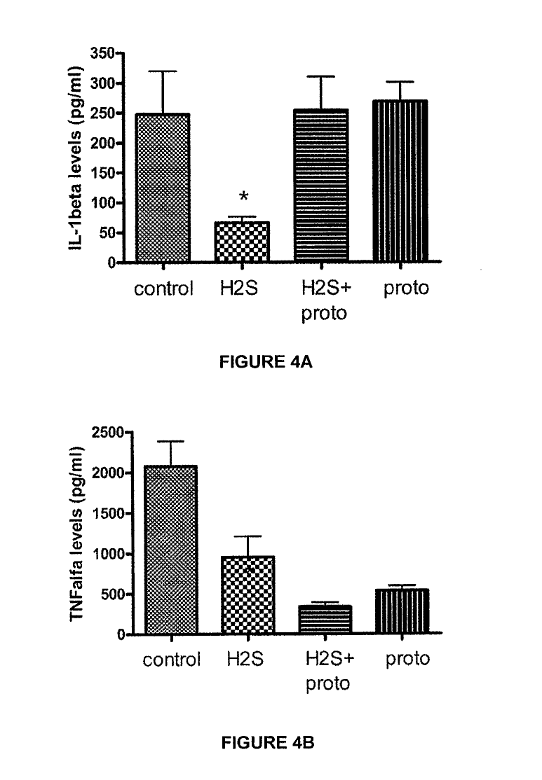

FIG. 4 demonstrates the in vivo anti-inflammatory effects of liquid sulfide pretreatment in mice subjected to bacterial LPS. FIG. 4A is a graph showing IL-1β production by mice treated with bacterial LPS following pretreatment with control vehicle, liquid sulfide, Tin-protoporphyrin IX, or both liquid sulfide and Tin-protoporphyrin IX for 30 minutes. FIG. 4B is a graph showing TNFα production by mice treated with bacterial LPS following pretreatment with control vehicle, liquid sulfide, Tin-protoporphyrin IX, or both liquid sulfide and Tin-protoporphyrin IX for 30 minutes.

### DETAILED DESCRIPTION OF THE INVENTION

As used in the specification and appended claims, unless specified to the contrary, the following terms have the meaning indicated:

The term “biological material” refers to any living biological material, including cells, tissues, organs, and/or organisms, and any combination thereof. It is contemplated that the methods of the present invention may be practiced on a part of an organism (such as in cells, in tissue, and/or in one or more organs), whether that part remains within the organism or is removed from the organism, or on the whole organism. Moreover, it is contemplated in the context of cells and tissues, both homogenous and heterogeneous cell populations may be the subject of embodiments of the invention. The term “in vivo biological matter” refers to biological matter that is in vivo, i.e., still within or attached to an organism. Moreover, the term “biological matter” will be understood as synonymous with the term “biological material.” In certain embodiments, it is contemplated that one or more cells, tissues, or organs is separate from an organism. The terms “isolated” and “ex vivo” are used to describe such biological material. It is contemplated that the methods of the present invention may be practiced on in vivo and/or isolated biological material.

The cells treated according to the methods of the present invention may be eukaryotic or prokaryotic. In certain embodiments, the cells are eukaryotic. More particularly, in some embodiments, the cells are mammalian cells. Mammalian cells include, but are not limited to those from a human, monkey, mouse, rat, rabbit, hamster, goat, pig, dog, cat, ferret, cow, sheep, and horse.

Cells of the invention may be diploid but in some cases, the cells are haploid (sex cells). Additionally, cells may be polyploid, aneuploid, or anucleate. In particular embodiments, a cell is from a particular tissue or organ, such as one from the group consisting of: heart, lung, kidney, liver, bone marrow, pancreas, skin, bone, vein, artery, cornea, blood, small intestine, large intestine, brain, spinal cord, smooth muscle, skeletal muscle, ovary, testis, uterus, and umbilical cord. In certain embodiments, cells are characterized as one of the following cell types: platelet, myelocyte, erythrocyte, lymphocyte, adipocyte, fibroblast, epithelial cell, endothelial cell, smooth muscle cell, skeletal muscle cell, endocrine cell, glial cell, neuron, secretory cell, barrier function cell, contractile cell, absorptive cell, mucosal cell, limbus cell (from cornea), stem cell (totipotent, pluripotent or multipotent), unfertilized or fertilized oocyte, or sperm.

The terms “tissue” and “organ” are used according to their ordinary and plain meanings. Though tissue is composed of cells, it will be understood that the term “tissue” refers to an aggregate of similar cells forming a definite kind of structural material. Moreover, an organ is a particular type of tissue. In certain embodiments, the tissue or organ is “isolated,” meaning that it is not located within an organism.

In various embodiments, methods of the present invention are used to treat any type of organism, including but not limited to, mammals, reptiles, amphibians, birds, fish, invertebrates, fungi, plants, protests, and prokaryotes. In particular embodiments, a mammal is a marsupial, an insect, a primate, or a rodent. In other embodiments, an organism is a human or a non-human animal. In specific embodiments, an animal is a mouse, rat, cat, dog, horse, cow, rabbit, sheep, fruit fly, frog, worm, or human.

“Mammal” includes humans and both domestic animals such as laboratory animals and household pets, (e.g., cats, dogs, swine, cattle, sheep, goats, horses, and rabbits), and non-domestic animals such as wildlife and the like.

“Optional” or “optionally” means that the subsequently described event of circumstances may or may not occur, and that the description includes instances where said event or circumstance occurs and instances in which it does not.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutical composition” refers to a formulation of a compound and a medium generally accepted in the art for the delivery of the biologically active compound to mammals, e.g., humans. Such a medium includes all pharmaceutically acceptable carriers, diluents or excipients therefore.

“Prodrug” refers to a compound that may be converted under physiological conditions or by solvolysis to a biologically active compound of the present invention. Thus, the term “prodrug” refers to a metabolic precursor of a compound of the present invention that is pharmaceutically acceptable. A prodrug may be inactive when administered to a subject in need thereof, but is converted in vivo to an active compound. Prodrugs are typically rapidly transformed in vivo to yield the active compound, for example, by hydrolysis in blood. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam)). A discussion of prodrugs is also provided in Higuchi, T., et al., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

“Sulfide” refers to sulfur in its −2 valence state, either as H₂S or as a salt thereof (e.g., NaHS, Na₂S, etc.). “H₂S” is generated by the spontaneous dissociation of the chalcogenide salt and H₂S donor, sodium hydrosulfide (NaHS), in aqueous solution according to the equations:

NaHS→Na++HS⁻

2HS⁻ [symbol] H₂S+S₂⁻

HS⁻+H+ [symbol] H₂S.

While the embodiments of the present invention described herein are primarily directed to sulfur compounds, it is understood that in other embodiments, the present invention may be practiced using chalcogenides other than sulfur. In certain embodiments, the chalcogenide compound comprises sulfur, while in others it comprises selenium, tellurium, or polonium. In certain embodiments, a chalcogenide compound contains one or more exposed sulfide groups. In particular embodiments, it is contemplated that this chalcogenide compound contains 1, 2, 3, 4, 5, 6 or more exposed sulfide groups, or any range derivable therein. In particular embodiments, such a sulfide-containing compound is CS₂ (carbon disulfide).

In certain embodiments, the chalcogenide is a salt, preferably salts wherein the chalcogen is in a −2 oxidation state. Sulfide salts encompassed by embodiments of the invention include, but are not limited to, sodium sulfide (Na₂S), sodium hydrogen sulfide (NaHS), potassium sulfide (K₂S), potassium hydrogen sulfide (KHS), lithium sulfide (Li₂S), rubidium sulfide (Rb₂S), cesium sulfide (Cs₂S), ammonium sulfide ((NH₄)₂S), ammonium hydrogen sulfide (NH₄)HS, beryllium sulfide (BeS), magnesium sulfide (MgS), calcium sulfide (CaS), strontium sulfide (SrS), barium sulfide (BaS), and the like.

“Chalcogenide precursor” refers to compounds and agents that can yield a chalcogenide, e.g., hydrogen sulfide (H₂S), under certain conditions, such as upon exposure, or soon thereafter, to biological matter. Such precursors yield H₂S or another chalcogenide upon one or more enzymatic or chemical reactions. In certain embodiments, the chalcogenide precursor is dimethylsulfoxide (DMSO), dimethylsulfide (DMS), methylmercaptan (CH₃SH), mercaptoethanol, thiocyanate, hydrogen cyanide, methanethiol (MeSH), or carbon disulfide (CS₂). In certain embodiments, the chalcogenide precursor is CS₂, MeSH, or DMS. Compounds on the order of the size of these molecules are particularly contemplated (that is, within about 50% of their molecular weights).

“Chalcogenide” or “chalcogenide compounds” refers to compounds containing a chalcogen element, i.e., those in Group 6 of the periodic table, but excluding oxides. These elements are sulfur (S), selenium (Se), tellurium (Te) and polonium (Po). Specific chalcogenides and salts thereof include, but are not limited to: H2S, Na2S, NaHS, K2S, KHS, Rb2S, CS2S, (NH4)2S, (NH4)HS, BeS, MgS, CaS, SrS, BaS, H2Se, Na2Se, NaHSe, K2Se, KHSe, Rb2Se, CS2Se, (NH4)2Se, (NH4)HSe, BeSe, MgSe, CaSe, SrSe, PoSe and BaSe.

The invention disclosed herein is also meant to encompass metabolic products of the disclosed compounds. Such products may result from, for example, the oxidation, reduction, hydrolysis, amidation, esterification, and the like of the administered compound, primarily due to enzymatic processes. Accordingly, the invention includes compounds produced by a process comprising contacting a compound of this invention with a mammal for a period of time sufficient to yield a metabolic product thereof. Such products are typically identified by administering a radiolabelled compound of the invention in a detectable dose to an animal, such as rat, mouse, guinea pig, monkey, or to human, allowing sufficient time for metabolism to occur, and isolating its conversion products from the urine, blood or other biological samples.

“Therapeutically effective amount” refers to that amount of a compound of the invention that, when administered to a mammal, preferably a human, is sufficient to effect treatment, as defined below, of a disease or condition in the mammal, preferably a human. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest, e.g., tissue injury, in a mammal, preferably a human, having the disease or condition of interest, and includes: (i) preventing the disease or condition from occurring in a mammal, in particular, when such mammal is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition. As used herein, the terms “disease,” “disorder,” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The present invention is based, on part, on the surprising discovery that administration of a combination of nitric oxide and sulfide to a cell results in reduced cytotoxicity or undesired side-effects as compared to administration of either nitric oxide or sulfide alone. Thus, the present invention provides methods of reducing cytotoxicity or undesired side-effects associated with administration of either nitric oxide or sulfide to biological material, e.g., cells, tissues, organs, organisms, and animals, which comprise administering either nitric oxide or sulfide in combination with the other.

According to the present invention, the combination of NO and sulfide counteracts, or neutralizes, undesirable pharmacological actions of NO or sulfide, including those that: i) exert harmful effects in mammals exposed thereto; or ii) antagonize, impede, reverse, or prevent the beneficial pharmacological or pharmaceutical effects of either NO, sulfide, or the combination thereof in mammals. These actions are known to those skilled in the art as “side effects” of drugs, meaning that the undesirable pharmacological actions of NO or sulfide are unwanted because they render less effective their known beneficial pharmacological or pharmaceutical actions. To the extent that NO and sulfide mitigate the side effects of pharmaceutical use of NO or sulfide, while preserving their beneficial effects, the instant invention contemplates the enhanced efficacy in mammals in need of NO or sulfide therapy that is derived from combining NO and sulfide as a pharmaceutical intervention.

In addition, according to certain aspects of the present invention, it is contemplated that combinations of nitric oxide and sulfide have increased biological and therapeutic activity in the treatment and prevention of various diseases and conditions presently treated with either nitric oxide or sulfide. In certain embodiments, the combination of nitric oxide and sulfide may have either additive or synergistic effects, e.g., in protecting cells and tissue from injury due to exposure to ischemic or hypoxic conditions.

Accordingly, the present invention includes improved methods of treating diseases and disorders previously treated with nitric oxide, which comprise administering nitric oxide in combination with sulfide. Further, the present invention provides improved methods of enhancing cell survival, inducing stasis, or protecting cells or tissue from injury due to hypoxia or ischemia, which comprise administering sulfide in combination with nitric oxide. The invention further includes compositions comprising both nitric oxide and sulfide, as well as methods and devices for the preparation and administration of combinations of nitric oxide and sulfide to a subject.

The example described herein demonstrates that a liquid pharmaceutical composition of hydrogen sulfide (liquid sulfide) provides protective benefits and reduces the cytotoxic effects of nitric oxide (NO) byproducts, s-nitrosoglutathione (GSNO) and peroxynitrite (ONOO—), a reactive cytotoxic oxidant species that is injurious to cells.

Without wishing to be bound to any particular theory, it is hypothesized that hydrogen sulfide may exert its protective effect by acting as a scavenger molecule to reduce or modify the effects of free radicals produced by nitric oxide. H₂S was previously shown to ‘scavenge’ peroxynitrite (ONOO—) (see: Halliwell and Whiteman, Methods Enzymol. (1999) 301:333-342). Thus, H₂S may inhibit the toxic effects of NO or its byproducts (e.g., peroxynitrite) (see Whiteman et al., Journal of Neurochemistry, 2004, 90, 765-768) to provide either or both a benefit to the pharmacological actions of NO or a reduction in the deleterious effects of its byproducts.

It is well known in the art that sulfides are unstable compounds and produce oxidation products. As used herein, “oxidation product” refers to products that result from sulfide chemical transformation, including, e.g., sulfite, sulfate, thiosulfate, polysulfides, dithionate, polythionate, and elemental sulfur. It is understood that nitric oxide may act to stabilize sulfide oxidation products.

Accordingly, it is understood in view of the present invention that the combination of nitric oxide and hydrogen sulfide may act to modify the effects of reaction products of nitric oxide and sulfide and thus confer the protective effect observed upon cotreatment with nitric oxide and sulfide. The present invention contemplates that hydrogen sulfide may be administered in combination with a therapeutic amount of nitric oxide to reduce an undesired side-effect of nitric oxide. Similarly, nitric oxide may be administered in combination with a therapeutic amount of sulfide to reduce an undesired side-effect of sulfide. Accordingly, in certain embodiments, the present invention also includes methods of using combinations of either nitric oxide or sulfide as well as compositions comprising either nitric oxide or sulfide.

### A. Methods of use of Nitric Oxide and Sulfide Combinations

The present invention, by reducing cytotoxicity or other undesirable side-effects associated with the administration of nitric oxide or sulfide to biological material, provides improved methods of treating or preventing diseases and disorders treated with either nitric oxide or sulfide. In addition, the present invention similarly provides improved methods of enhancing the survivability of biological material under hypoxic or ischemic conditions, as well as related methods of protecting biological material from injury due to hypoxia or ischemia and inducing stasis. These methods comprise providing a combination of nitric oxide and sulfide to the biological material.

Combinations of nitric oxide and sulfide may be administered to biological material at the same time, sequentially in any order, or both. For example, in certain embodiments, biological material is pretreated with an amount of either sulfide or nitric oxide sufficient to confer a protective effect, and then subsequently treated with a therapeutically effective amount of either nitric oxide or sulfide, respectively. In other embodiments, both nitric oxide and sulfide are provided to biological material, e.g., a mammal, at the same time. They may be provided at the same time by coadministration of separate formulations of each of nitric oxide and sulfide, or by administration of a formulation comprising both nitric oxide and sulfide. Because NO and sulfide may, under certain circumstances (Whiteman et al., 2006), react chemically with each other, in certain embodiments of the invention, NO and sulfide are separately formulated (gas or liquid) and then administered concomitantly.

Combinations of nitric oxide may be administered in any combination of gas and liquid forms of either or both nitric oxide and sulfide. NO and H₂S are gases at standard temperature and pressure (STP). NO is soluble in water up to a concentration of about 2 millimolar (2 mM) at STP. Sulfide is soluble in water up to over 100 millimolar (100 mM) at STP. With these properties, both NO and sulfide may be administered to mammals in need of therapeutic intervention either as a gas, e.g., by inhalation or ventilation, or as a liquid, e.g., by parenteral (e.g., intravenous, intraarterial), oral, topical or sublingual dosage forms.

Accordingly, in various embodiments, both nitric oxide and sulfide are administered as gases or both nitric oxide and sulfide are administered as liquid formulations. The nitric oxide and sulfide may be present in the same gas or liquid formulation, or they may be in separate gas or liquid formulations. In another embodiment, nitric oxide is administered as a gas, and sulfide is administered as a liquid formulation. In another embodiment, nitric oxide is administered as a liquid formulation, and sulfide is administered as a gas.

In specific exemplary embodiments, administering a combination of nitric oxide and sulfide includes: a) administering by inhalation/ventilation a mixture of NO and sulfide gases; b) administering NO gas by inhalation/ventilation and, concomitantly, sulfide by parenteral (e.g., intravenous) administration of a liquid sulfide pharmaceutical composition; c) administering a liquid NO pharmaceutical composition by parenteral injection and, concomitantly, H₂S gas by inhalation/ventilation; d) administering a liquid NO pharmaceutical composition and, concomitantly, a liquid sulfide pharmaceutical composition; and e) administering by a nebulizer a liquid NO pharmaceutical composition and, concomitantly or sequentially a liquid sulfide pharmaceutical composition. Various gas and liquid formulations of nitric oxide and sulfide are known in the art and described herein.

### 1. Methods of Use of Nitric Oxide

In certain embodiments, methods, compositions, and devices of the present invention are used to treat or prevent any of a variety of diseases and disorders that benefit from treatment with nitric oxide, or a precursor, prodrug, analog, derivative, or metabolic or degradation product thereof. In particular embodiments, the methods of the present invention may be used to modulate biological pathways regulated or affected by nitric oxide, including those described herein. Nitric oxide is a ubiquitous cell signaling molecule, and multiple forms of NO have been described, specific to particular organ systems and even to individual species.

Nitric oxide activity is associated with numerous biological pathways and/or effects, including maintaining or regulating blood pressure, such as by lowering mean arterial blood pressure or pulmonary artery pressure, causing vasodilation, providing hypoxemia relieving effects, regulating communication of the endothelial lining of blood vessels communicated with the underlying vascular smooth muscle, and the like. Additionally, nitric oxide plays a role in neurotransmission, stimulation of the immune responses, modulation of the hair cycle, penile erections, ischaemia-reperfusion injuries, regulating mitochondrial respiration, affecting angiogenesis, cell death, e.g., such as tumor or neuronal cell death, and increasing cyclic guanosine monophosphate (cGMP) production.

NO has many physiological actions in mammals, including, but not limited to, vasodilatation, cytoprotection, and pro-inflammation. Additional biological activities of nitric oxide include counteracting thromboxane, affecting platelet function such as by inhibiting platelet aggregation through stimulation of guanylate cyclase and inhibiting platelet activation, causing the release of prostanoids, stimulating prostanoids through activation of cyclooxygenase, reducing myocardial contractility, attenuating inotropic response, reducing cardiac lactate accumulation by forming cGMP, dilating coronary arteries, regulating hypoxia-inducible factor la, a transcription factor that is a key regulator of oxygen homeostasis, suppressing ventricular fibrillation, producing oxygen free radicals, contributing to systemic hypotension of septic shock, mediating neuronal plasticity, mediating the relaxation of the oesophageal and pyloric sphincters in the gut, regulating urogenital function, stimulating renin release in the kidneys, improving oxygenation to the lungs, reducing shunt perfusion in the lung, and the like.

An additional example of a biological reaction associated with nitric oxide is S-nitrosation (or S-nitrosylation), the covalent attachment of a nitrogen monoxide group to the thiol side chain of cysteine within proteins. S-nitrosylation has emerged as a mechanism for dynamic, post-translational regulation of most or all main classes of proteins. For example, nitric oxide may stimulate or inhibit cysteine-containing receptor proteins, including serotonin receptors, adrenergic receptors, NMDA receptors, ryanodine receptors, muscarinic receptors, and kinin receptors, and may modify the function of cysteine-containing non-receptor proteins including hemoglobin, NFκB, AP1, ras, Na⁺ channels, Ca²⁺ channels, K⁺ channels, and prion protein.

Nitric oxide and related molecules are also toxic to bacteria and other human pathogens, such as when produced by macrophages as part of an immune response.

NO gas (10-80 parts per million mixed into air) is approved by the U.S. Food and Drug Administration (FDA) for use in the treatment of neonatal respiratory distress and may be useful in treating other human and animal diseases or injuries, including myocardial infarction, stroke, hemorrhage, and major surgery. It is thought to be effective in newborn children experiencing respiratory distress in part because it causes vasodilatation of the lung vasculature.

The methods and compositions of the present invention may be used to treat or prevent a variety of diseases and disorders, including any disease or disorder that has been treated using any of a gaseous form of nitric oxide, a liquid nitric oxide composition or any medically applicable useful form of nitric oxide, including any described in U.S. Pat. No. 6,103,275.

Diseases, disorders, and conditions that may benefit from treatment with, or are associated with, nitric oxide, nitric oxide precursors, analogs, or derivatives thereof, include elevated pulmonary pressures and pulmonary disorders associated with hypoxemia (e.g., low blood oxygen content compared to normal, i.e., a hemoglobin saturation less than 95% and a Pa_{O2} less than 90 in arterial blood in someone breathing room air) and/or smooth muscle constriction, including pulmonary hypertension, acute respiratory distress syndrome (ARDS), diseases of the bronchial passages such as asthma and cystic fibrosis, other pulmonary conditions including chronic obstructive pulmonary disease, adult respiratory distress syndrome, high-altitude pulmonary edema, chronic bronchitis, sarcoidosis, cor pulmonale, pulmonary embolism, bronchiectasis, emphysema, Pickwickian syndrome, and sleep apnea.

Additional examples of conditions associated with nitric oxide or nitric oxide related treatments include cardiovascular and cardio-pulmonary disorders, such as angina, myocardial infarction, heart failure, hypertension, congenital heart disease, congestive heart failure, valvular heart disease, and cardiac disorders characterized by, e.g., ischemia, pump failure and/or afterload increase in a patient having such disorder, and artherosclerosis. Nitric oxide related treatments may also find use in angioplasty.

Additional examples include blood disorders, including those blood disorders ameliorated by treatment with NO or related molecules, i.e., where NO would change the shape of red blood cells to normal or restore their function to normal or would cause dissolution of blood clots. Examples of blood disorders include, e.g., sickle cell disease and clotting disorders including disseminated intravascular coagulation (DIC), heart attack, stroke, and Coumadin-induced clotting caused by Coumadin blocking protein C and protein S, and platelet aggregation;

Additional examples include such conditions as hypotension, restenosis, inflammation, endotoxemia, shock, sepsis, stroke, rhinitis, and cerebral vasoconstriction and vasodilation, such as migraine and non-migraine headache, ischemia, thrombosis, and platelet aggregation, including preservation and processing of platelets for transfusions and perfusion technologies, diseases of the optic musculature, diseases of the gastrointestinal system, such as reflux esophagitis (GERD), spasm, diarrhea, irritable bowel syndrome, and other gastrointestinal motile dysfunctions, depression, neurodegeneration, Alzheimer's disease, dementia, Parkinson's disease, stress and anxiety.

Nitric oxide and nitric oxide related treatments may also be useful in suppressing, killing, and inhibiting pathogenic cells, such as tumor cells, cancer cells, or microorganisms, including but not limited to pathogenic bacteria, pathogenic mycobacteria, pathogenic parasites, and pathogenic fungi. Examples of microorganisms include those associated with a respiratory infection within the respiratory tract.

Uses and potential uses of NO contemplated by the present invention include: prevention of localized tissue damage (see U.S. Pat. No. 6,255,277), as an antibacterial (see US 2003/0228564; US 2002/0155164), as an anti-inflammatory, or used in combination as an adjuvant to enhance anti-inflammatory properties of glucocorticoids (see PCT application WO 2004/087212), in wound healing (see US 2004/0009238), blood pressure regulation, cardiovascular disease, gastrointestinal disease, central nervous system disorders, diabetes, reproductive disorders, bladder and kidney diseases, dermatological problems (see U.S. Pat. No. 6,103,275), in tendinopathy (see US 2005/0171199), in nail infections (see PCT application WO 03/013489) and anal disorders (see U.S. Pat. No. 5,504,117).

In certain embodiments, the present invention provides methods of treating or preventing any of these diseases or disorders, which methods comprise administering a therapeutically effective amount of nitric oxide (or precursor, prodrug, analog, derivative, or metabolic product thereof) to a patient in combination with a sulfide. In other embodiments, the present invention also includes related methods of modulating a biological activity associated by nitric oxide, comprising contacting biological matter with an effective amount of nitric oxide in combination with sulfide.

### 2. Methods of Use of Sulfide

In certain embodiments, methods, compositions, and devices of the present invention are used for purposes associated with the administration of sulfide to biological matter.

Sulfide has many physiological actions in mammals, including, but not limited to, vasodilatation, cytoprotection, metabolic depression (or stasis), and anti-inflammation. When administered either parenterally or by inhalation or ventilation to mammals, sulfide reduces injury and enhances survivability in the setting of myocardial infarction, cardiac surgery, lethal hemorrhage, cerebral and hepatic ischemia, and lethal hypoxia. Sulfide may reduce injury or enhance survivability in similar or other human diseases or injuries.

Accordingly, the present invention provides a variety of methods for enhancing the survivability of, and/or reducing damage to, biological material under ischemic or hypoxic conditions, which involve contacting the biological material with an effective amount of sulfide in combination with nitric oxide. In various embodiments, the biological material is contacted with either or both nitric oxide and sulfide prior to being subjected to ischemic or hypoxic conditions. In other embodiments, the biological material is contacted with either or both nitric oxide and sulfide during all or part of the time of exposure to ischemic or hypoxic conditions. In another related embodiment, the biological material is contacted with either or both nitric oxide and sulfide both prior to and during all or part of the time of exposure to ischemic or hypoxic conditions. In another embodiment, the biological material is contacted with either or both nitric oxide and sulfide after reperfusion of the ischemic or hypoxic biological matter. It is understood that the time during which biological material is contacted with sulfide may be different, overlapping, or the same time period during which it is contacted with nitric oxide.

“Enhancing survivability” generally refers to either or both of (1) increasing the likelihood that a biological material will survive exposure to ischemic or hypoxic conditions and (2) extending the duration of time that a biological material survives exposure to ischemic or hypoxic conditions. In particular embodiments, by contacting the biological material with sulfide and nitric oxide, the likelihood that the biological material will survive being exposed to hypoxic or ischemic conditions is increased by at least 25%, at least 50%, at least 100%, at least 200%, at least 300%, at least 400%, at least 500%, or at least 1000%. In other embodiments, by contacting the biological material with sulfide and nitric oxide, the duration of time that the biological material will survive during or after exposure to ischemic or hypoxic conditions is increase by at least 25%, at least 50%, at least 100%, at least 200%, at least 300%, at least 400%, at least 500%, or at least 1000%.

In other embodiments, the compositions and methods of the present invention may be used to induce biological material to enter a hypometabolic state wherein the biological material is alive but is characterized by one or more of: (1) at least a 50% reduction in the rate or amount of carbon dioxide production by the biological matter; and (2) at least a 50% reduction in the rate or amount of oxygen consumption by the biological matter. In another embodiment, the compositions and methods of the present invention may be used to induce biological material to enter a hypometabolic state wherein the biological material is alive but is characterized by one or more of: (1) a less than 50% reduction in the rate or amount of carbon dioxide production by the biological matter; and (2) a less than 50% reduction in the rate or amount of oxygen consumption by the biological matter. Any assay to measure oxygen consumption or carbon dioxide production may be employed, and a typical assay will involve utilizing a closed environment and measuring the difference between the oxygen put into the environment and oxygen that is left in the environment after a period of time. Typically, any reduction in the metabolic activity of a biological material is reversible.

According to various embodiments of the methods of the present invention, a hypometabolic state is induced by treating biological material with an amount of sulfide and nitric oxide that induces hypometabolism directly itself or, alternatively, by treating biological material with an amount of sulfide and nitric oxide that does not itself induce hypometabolism, but instead, promotes or enhances the ability of or decreases the time required for the biological material to enter a hypometabolic state in response to another stimuli, such as, but not limited to, an injury, a disease, hypoxia, reduced temperature conditions, excessive bleeding, or treatment with one or more other active compounds (as defined herein).

It is understood that the particular applications of the methods of the present invention vary depending upon the type of biological material being treated, i.e., cells, tissues, organs, or organisms, and the particular ischemic or hypoxic conditions under which the biological material is exposed. Specific embodiments related to particular types of biological material and ischemic or hypoxic conditions are described further herein.

Ischemic and hypoxic conditions may be accidental or purposeful, and ischemic and hypoxic conditions may result from a variety of biological and environmental factors. For example, in the context of mammals, ischemic and hypoxic conditions include those resulting from injury or disease, as well as those resulting from cryopreservation techniques. In the context of tissues and organs, hypoxic and ischemic conditions may arise during procedures to preserve organs or tissues prior to transplant or grafting. Similarly, cells may be exposed to hypoxic or ischemic conditions during cryopreservation.

Specific examples of conditions leading to ischemia and hypoxia include, but are not limited to, when oxygen concentrations are reduced in the environment (hypoxia or anoxia, such as at high altitudes or under water); when biological material is incapable of receiving oxygen (such as during ischemia), which can be caused by: i) reduced blood flow to organs (e.g., heart, brain, and/or kidneys) as a result of blood vessel occlusion (e.g., myocardial infarction and/or stroke); ii) extracorporeal blood shunting as occurs during heart/lung bypass surgery (e.g., “pumphead syndrome” in which heart or brain tissue is damaged as a result of cardiopulmonary bypass); or iii) blood loss due to trauma (e.g., hemorrhagic shock or surgery); hypothermia, wherein the biological material is subjected to sub-physiological temperatures, due to exposure to a cold environment or a state of low temperature of the biological material, such that it is unable to maintain adequate oxygenation; hyperthermia, wherein the biological material is subjected to supra-physiological temperatures, due to exposure to a hot environment or a state of high temperature of the biological material such as by a malignant fever; and conditions of excess heavy metals, such as iron disorders (genetic as well as environmental) such as hemochromatosis, acquired iron overload, sickle-cell anemia, juvenile hemochromatosis African siderosis, thalassemia, porphyria cutanea tarda, sideroblastic anemia, iron-deficiency anemia and anemia of chronic disease.

It will be further appreciated that the length of time with which biological material is contacted with sulfide and nitric oxide will vary depending upon the type of biological material, the desired outcome, the particular type of injury or disease, and the particular type of ischemic challenge faced by the biological material. For example, inducing a hypometabolic state with respect to a whole animal and with respect to cells or tissues may require different lengths of treatment. In addition, with respect to human subjects, e.g., subjects undergoing a surgical treatment, treatment for a hemorrhagic shock, or treatment for a hyperproliferative disorder, maintaining the subject in a hypometabolic state for 12, 18, or 24 hours is generally contemplated. With respect to non-human animal subjects, e.g. non-human animals shipped or stored for commercial purposes, maintaining the subject in a hypometabolic state for a period of 2 or 4 days, 2 or 4 weeks, or longer is contemplated.

In addition, it is also understood that the amount of sulfide and nitric oxide required will vary depending upon whether the biological material is also being treated with another stimuli, i.e., an agent or conditions that induces a hypometabolic state. In such circumstances, the biological material may be contacted with sulfide and nitric oxide for all or only a part of the duration of time the method is performed, in order to, e.g., enhance survivability of the biological material or protect it from ischemic damage.

a. In Vivo Methods

In certain embodiments, the present invention provides methods of enhancing the survivability of biological materials, including, e.g., organisms (including mammals), that are subjected to ischemic or hypoxic conditions. In related embodiments, the present invention provides methods of preventing or reducing damage to biological materials, including, e.g., mammals, including cell or tissue injuries resulting from ischemia or hypoxia. It is understood that a whole biological material or only a portion thereof, e.g., a particular organ, may be subjected to ischemic or hypoxic conditions. However, in particular embodiments, the whole biological material may be subjected to ischemic conditions, for example, to assist in the preservation of an organism.

In particular embodiments, the ischemic or hypoxic conditions are the result of an injury or disease suffered by an organism. Accordingly, the present invention provides methods of enhancing survivability of an organism suffering from any disease or injury, including those described below, which methods comprise contacting the organism with an effective amount of sulfide and nitric oxide. Examples of specific diseases that can induce ischemia or hypoxia include, but are not limited to, tumors, heart diseases, and neurological diseases. Examples of specific injuries that can result in ischemic or hypoxic conditions include, but are not limited to, external insults, such as burns, cutting wounds, amputations, gunshot wounds, or surgical trauma. In addition, injuries can also include internal insults, such as stroke or heart attack, which result in the acute reduction in circulation. Other injuries include reductions in circulation due to non-invasive stress, such as exposure to cold or radiation, or a planned reduction in circulation, e.g., during heart surgery. On a cellular level, such injuries often result in exposure of cells, tissues, and/or organs to hypoxic conditions, thereby resulting in induction of programmed cell death, or “apoptosis.” Systemically, these injuries can lead to the induction of a series of biochemical processes, such as clotting, inflammation, hypotension, and may give rise to shock, which if it persists may lead to organ dysfunction, irreversible cell damage and death. In a specific scenario, where medical attention is not readily available, such contacting with sulfide and nitric oxide, alternatively in conjunction with reduction in the temperature of the tissue, organ or organism, can “buy time” for the subject, either by bringing medical attention to the subject, or by transporting the subject to the medical attention.

The present invention also contemplates methods for inducing tissue regeneration and wound healing by prevention/delay of biological processes that may result in delayed wound healing and tissue regeneration. In this context, in scenarios in which there is a substantial wound to a limb or organism, the contacting with sulfide and nitric oxide, in vivo or ex vivo, alone or in combination with another active compound or reduced oxygen conditions, alternatively in conjunction with reduction in the temperature of the tissue, organ or organism, aids in the wound healing and tissue regeneration process by managing the biological processes that inhibit healing and regeneration.

In certain embodiments, methods of the present invention can be implemented to enhance survivability and prevent ischemic injury resulting from cardiac arrest or stroke. Accordingly, in one embodiment, the present invention includes methods of enhancing survivability or reducing ischemic injury in a patient suffering from or at risk of cardiac arrest or stroke, comprising providing an effective amount of sulfide and nitric oxide to the patient before, after, or both before and after myocardial infarction, cardiac arrest or stroke.

In certain embodiments, methods of the present invention include pre-treating a biological material, e.g., a patient, prior to an ischemic or hypoxic injury or disease insult. These methods can be used when an injury or disease with the potential to cause ischemia or hypoxia is scheduled or elected in advance, or predicted in advance to likely occur. Examples of such situations include, but are not limited to, major surgery where blood loss may occur spontaneously or as a result of a procedure, cardiopulmonary bypass in which oxygenation of the blood may be compromised or in which vascular delivery of blood may be reduced (as in the setting of coronary artery bypass graft (CABG) surgery), or in the treatment of organ donors prior to removal of donor organs for transport and transplantation into a recipient in need of an organ transplant. Other examples include, but are not limited to, medical conditions in which a risk of injury or disease progression is inherent (e.g., in the context of unstable angina, following angioplasty, bleeding aneurysms, hemorrhagic strokes, following major trauma or blood loss), or in which the risk can be diagnosed using a medical diagnostic test. In one embodiment, the ischemia or hypoxia is not myocardial ischemia or hypoxia. In another embodiment, the ischemia or hypoxia is not due to myocardial infarction. In another embodiment, the biological material is not a myocyte or heart tissue.

In certain embodiments, exposure to sulfide and nitric oxide enhances survivability or reduces damage when exposure occurs before the injury or disease insult. In other embodiments, exposure to sulfide and nitric oxide, enhances survivability or reduces damage when exposure occurs after the onset or detection of the injurious or disease insult, and either before or after the injury or disease causes ischemia or hypoxia.

In certain embodiments, the present invention includes methods of enhancing survivability of a mammal undergoing a surgery. In a related embodiment, a method is provided for protecting a mammal from suffering ischemic injury or cellular damage resulting from a surgery. These methods comprise providing to the mammal an effective amount of sulfide and nitric oxide prior to, during, or both prior to and during the surgery. The surgery may be elective, planned, or emergency surgery, such as, e.g., cardiopulmonary surgery. The sulfide and nitric oxide may be administered by any means available in the art, including, e.g., by inhalation or intravenously.

The invention has particular importance with respect to the risk of ischemic injury from emergency surgical procedures, such as thoracotomy, laparotomy, and splenic transection. Therefore, it includes methods of enhancing survivability or reducing or preventing ischemic injury in a patient undergoing an emergency surgery, comprising providing an effective amount of sulfide and nitric oxide, to the patient before surgery, after surgery, or both before and after surgery.

In another embodiment, the present invention includes a method of enhancing survivability of a mammal suffering from a disease or adverse medical condition that causes ischemia or hypoxia within a region of the mammal. A related embodiment includes a method of protecting a mammal from suffering ischemic injury or cellular damage from a disease or adverse medical condition. These methods typically comprise providing to the mammal an effective amount of sulfide and nitric oxide, prior to, after, or both prior to and after, the onset of or progression of the disease or adverse medical condition. This embodiment may be used in the context of a variety of different diseases and adverse medical conditions, including, e.g., unstable angina, post-angioplasty, aneurysm, hemorrhagic stroke or shock, trauma, and blood loss.

In specific embodiments, the invention concerns methods of preventing an organism, such as a mammal, from bleeding to death or suffering irreversible tissue damage as a result of bleeding by providing to the mammal an effective amount of sulfide and nitric oxide. In certain additional embodiments, the organism may go into hemorrhagic shock but not die from excessive bleeding. The terms “bleeding” and “hemorrhaging” are used interchangeably to refer to any discharge of blood from a blood vessel. It includes, but is not limited to, internal and external bleeding, bleeding from an injury (which may be from an internal source, or from an external physical source such as from a gunshot, stabbing, physical trauma, etc.).

Moreover, additional embodiments of the invention concern enhancing survivability and preventing irreversible tissue damage from blood loss or other lack of oxygenation to cells or tissue, such as from lack of an adequate blood supply. This may be the result of, for example, actual blood loss, or it may be from conditions or diseases that cause blockage of blood flow to cells or tissue, that reduce blood pressure locally or overall in an organism, that reduce the amount of oxygen is carried in the blood, or that reduces the number of oxygen carrying cells in the blood. Conditions and diseases that may be involved include, but are not limited to, blood clots and embolisms, cysts, growths, tumors, anemia (including sickle cell anemia), hemophilia, other blood clotting diseases (e.g., von Willebrand's Disease, ITP), and atherosclerosis. Such conditions and diseases also include those that create essentially hypoxic or anoxic conditions for cells or tissue in an organism because of an injury, disease, or condition.

In one embodiment, the present invention provides methods to enhance the survivability of and prevent injury or damage to biological material undergoing hemorrhagic shock, which include contacting the biological material subjected to shock with sulfide and nitric oxide. In a certain embodiment, these methods are used to preserve a patient's vital organs and life. Hemorrhagic shock is a life-threatening condition in which inadequate perfusion to sustain the physiologic needs of organs or tissues occurs. The resulting inadequate oxygenation of tissues and organs can result in significant tissue and organ damage, and frequently death. Hemorrhagic shock may result from inadequate blood volume (hypovolaemic shock), inadequate cardiac function (cardiogenic shock), or inadequate vasomotor tone, also referred to as distributive shock (neurogenic shock, septic shock, anaphylactic shock). Specific conditions associated with hemorrhagic shock include, e.g., sepsis, blood loss, impaired autoregulation, and loss of autonomic tone, spontaneous hemorrhage (e.g., gastrointestinal bleeding, childbirth), surgery, and other causes. Most frequently, clinical hemorrhagic shock is caused by an acute bleeding episode with a discrete precipitating event. Less commonly, hemorrhagic shock may be seen in chronic conditions with subacute blood loss.

In certain embodiments, the present invention includes a method of contacting a patient suffering from an acute injury and at risk of or in a state of hemorrhagic shock with an effective amount of sulfide and nitric oxide, within one hour of the injury. This method allows for the patient to be transported to a controlled environment (e.g., surgery), where the initial cause of the shock can be addressed, and then the patient can be brought back to normal function in a controlled manner. For this indication, the first hour after injury, referred to as the “golden hour,” is crucial to a successful outcome. Stabilizing the patient in this time period is the major goal, and transport to a critical care facility (e.g., emergency room, surgery,) where the injury can be properly addressed.

In certain embodiments, the present invention provides methods related to treating cancer and other hyperproliferative diseases. Cancer is a leading cause of mortality in industrialized countries around the world. The most conventional approach to the treatment of cancer is by administering a cytotoxic agent or cytotoxic agents to the cancer patient (or treatment ex vivo of a tissue) such that the agent or agents have a more lethal effect on the cancer cells than normal cells. The higher the dose or the more lethal the agent, the more effective it is in killing cancer cells. However, by the same token, such agents are all that more toxic (and sometimes lethal) to normal cells. Hence, chemo- and radiotherapy are often characterized by severe side effects, some of which are life threatening, e.g., sores in the mouth, difficulty swallowing, dry mouth, nausea, diarrhea, vomiting, fatigue, bleeding, hair loss and infection, skin irritation and loss of energy (Curran, 1998; Brizel, 1998).

In one embodiment, the present invention contemplates the use of sulfide and nitric oxide to protect normal tissues of a patient being treated for cancer or another hyperproliferative disease, thereby reducing the potential impact of chemo- or radiotherapy on those tissues, and enhancing survivability of the patient. These methods permit the use of higher doses of chemo- and radiotherapy, thereby increasing the anti-cancer effects of these treatments. Recent studies suggest that transient and reversible lowering of the core body temperature, or “hypothermia,” may lead to improvements in the fight against cancer. Hypothermia of 28° C. was recently found to reduce radiation, doxorubicin-and cisplatin-induced toxicity in mice. The cancer fighting activity of these drugs/treatments was not compromised when administered to cooled animals; rather, it was enhanced, particularly for cisplatin (Lundgren-Eriksson et al., 2001). Methods of the invention further include contacting a patient with sulfide and nitric oxide in combination with an immunotherapeutic agent.

The methods of the present invention may be used in the treatment of neurodegenerative diseases associated with ischemia or hypoxia. Neurodegenerative diseases are characterized by degeneration of neuronal tissue, and are often accompanied by loss of memory, loss of motor function, and dementia. With dementia, intellectual and higher integrative cognitive faculties become more and more impaired over time. It is estimated that approximately 15% of people 65 years or older are mildly to moderately demented. Neurodegenerative diseases include Parkinson's disease; primary neurodegenerative disease; Huntington's Chorea; stroke and other hypoxic or ischemic processes; neurotrauma; metabolically induced neurological damage; sequelae from cerebral seizures; hemorrhagic shock; secondary neurodegenerative disease (metabolic or toxic); Alzheimer's disease and other memory disorders; or vascular dementia, multi-infarct dementia, Lewy body dementia, or neurodegenerative dementia. The present invention provides methods of preventing tissue damage from neurological diseases associated with ischemia, comprising administering sulfide and nitric oxide to a patient suffering from such a disease or condition.

In yet another embodiment, the methods of the present invention are used to treat a mammal with extreme hypothermia. The methods and compositions of the present invention are useful for enhancing survivability of an organism subjected to extreme hypothermia. In one embodiment, these methods include enhancing survivability of an organism by inducing mild hypothermia in the organism in combination with contacting the organism with sulfide and nitric oxide. Hypothermia can be mild, moderate or profound. Mild hypothermia comprises achievement of a core body temperature of approximately between 0.1 and 5 degrees Celsius below the normal core body temperature of the mammal. The normal core body temperature of a mammal is usually between 35 and 38 degrees Celsius. Moderate hypothermia comprises achievement of a core body temperature of approximately between 5 and 15 degrees Celsius below the normal core body temperature of the mammal. Profound hypothermia comprises achievement of a core body temperature of approximately between 15 and 37 degrees Celsius below the normal core body temperature of the mammal.

Mild hypothermia is known in the art to be therapeutically useful and effective in both non-human mammals and in humans. The therapeutic benefit of mild hypothermia has been observed in human clinical trials in the context of out-of-hospital cardiac arrest. Exposure of humans to mild hypothermia in the context of cardiac arrest results in a survival advantage and an improved neurological outcome compared to standard of care with normothermia, or absence of mild hypothermia (Bernard et al., 2002; The Hypothermia After Cardiac Arrest Study Group et al. 2002).

In one embodiment, a method of the present invention provides that patients with extreme hypothermia are administered or exposed to sulfide and nitric oxide and then gradually restored to normal temperature while withdrawing, in a controlled fashion, the sulfide and nitric oxide. In this way, sulfide and nitric oxide buffers the biological systems within the subject so that they may be initiated gradually without shock (or harm) to the subject. Ideally, the patient will be stabilized in terms of heart rate, respiration and temperature prior to effecting any change. Once stable, the ambient environmental temperature will be increased, again gradually. This may be accomplished simply by removing the subject from the hypothermic conditions. A more regulated increase in temperature may be affected by adding successive layers of clothing or blankets, by use of a thermal wrap with gradual increase in heat, or if possible, by placing the subject in chamber whose temperature may be gradually increased.

The vital signs of the subject may be monitored over the course of the temperature increase. Also, in conjunction with increasing the temperature, sulfide and nitric oxide is removed from the subject's environment. Both heat and sulfide and nitric oxide treatment are ceased at the appropriate endpoint, judged by the medical personnel monitoring the situation, but in any event at the time the subject's temperature and other vital signs return to a normal range. Continued monitoring following cessation of treatment is recommended for a period of at least 24 hrs.

In other embodiments, the methods of the present invention are used to treat hyperthermia. Under certain conditions, which can result from genetic, infectious, drug, or environmental causes, patients can loose homeostatic temperature regulation resulting in severe uncontrollable fever (hyperthermia). This can result in mortality or long-term morbidity, especially brain damage, if it is not controlled properly. The present invention provides methods of treating hyperthermia that involve contacting the patient with sulfide and nitric oxide to induce reduced metabolic activity and enhance survivability or reduce injury to potentially affected brain tissue. In particular embodiments, the patient is contacted for between about 6 and about 24 hours, during which time the source of the fever can be addressed. This treatment can be combined with whole-body temperature regulation, such as an ice bath/blanket/cooling system.

The present invention further demonstrates that sulfide has anti-inflammatory effects in viva Accordingly, the present invention includes methods of treating, reducing or preventing inflammation comprising administering a sulfide composition to biological matter, such as a mammal. These methods may include pre-treatment with sulfide before an inflammatory trigger or event, or they may include treatment with sulfide following an inflammatory trigger or event.

In particular embodiments, these methods may be used to treat or prevent inflammation resulting from a free radial or reactive oxygen species. In particular embodiments, the inflammation is caused by nitric oxide or a product thereof, such as GSNO or ONOO—.

These methods may be used to treat or prevent inflammation in mammals that results from a variety of triggers or event, including both acute events, such as contact with an allergen, and more long-term inflammation, such as that resulting from a transient or chronic inflammatory disease or disorder.

Various inflammatory-related diseases or disorders that may be treated by methods of the present invention include, e.g., multiple sclerosis, arthritis, rheumatoid arthritis, systemic lupus erythematosus, graft versus host disease, diabetes, psoriasis, progressive systemic sclerosis, scleroderma, acute coronary syndrome, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis.

b. Ex Vivo Methods

In certain embodiments, the methods of the present invention are used to enhance the survivability of ex vivo biological matter subjected to hypoxic or ischemic conditions, including, e.g., isolated cells, tissues and organs. Specific examples of such ex vivo biological material include platelets and other blood products, as well as tissues and organs to be transplanted.

In one embodiment, methods of the present invention may be used to enhance survivability of biological material in the laboratory or research context, for example when cell lines or laboratory organisms are purposefully subjected to hypoxic or ischemic conditions, e.g., during cryopreservation and storage.

The present invention can be extended to protecting cells in culture, which might otherwise die or be induced into apoptosis. According to the present invention, cells are exposed to sulfide and nitric oxide prior to and/or while in culture. Cells that can be cultured according to the invention include those that can eventually be placed back into a physiological context, i.e., those for subsequent transplant. Such cells include, but are not limited to, bone marrow cells, skin cells, stem cells, and epithelial cells. Also, some transplantable cells would greatly benefit from expansion in culture, thereby increasing the amount of material that can be introduced into the host. In one particular embodiment, the methods of the present invention are applied to epithelial cells from the gastrointestinal tract.

Furthermore, the invention extends to the culture of tumor cells. Culture of tumor cells is known to result in alteration of the phenotype and, in some cases, death. This makes tissue culture experiments on tumor cells highly unpredictable.

General cell culture techniques are well known to those of skill in the art. Examples of this knowledge can be found in Shaw (1996) and Davis (1994), both of which are incorporated by reference herein. General information and modifications of traditional cell culture techniques is also found in U.S. Pat. No. 5,580,781, which is incorporated by reference. Furthermore, techniques for culturing skin cells are described in U.S. Pat. No. 6,057,148, which is incorporated by reference. It is contemplated that these techniques, as well as others known to those of skill in the art, will be supplemented with media containing sulfide and/or nitric oxide or under conditions where they are exposed to nitric oxide and/or sulfide gas.

The invention also provides methods of enhancing the survivability of, or preserving, tissues and organs for transplant, which comprise contacting the tissue or organ with sulfide and nitric oxide. Initial contact can occur prior to removal from a donor or following removal from a donor. While there is a constant need for organ donors, a significant hurdle in providing those in need of an organ transplant with an organ is the limitations in current organ preservation techniques. Indeed, the primary cause of organ transplant failure for transplanted hearts in the first 30 days is ischemic-reperfusion injury. It is widely believed that a human heart must be transported within four hours for there to be any chance of the subsequent transplantation to be a success. Similarly, the maximum cold ischemic time allowed for liver is 12-24 hours, kidney is 48-72 hours, pancreas is 12-24 hours, and small intestine is 12 hours (Rager, 2004). Tissues useful for transplant include, but are not limited to, skin tissue. Organs useful for transplant include, but are not limited to, hearts, lungs, kidneys, livers, pancreas, small intestine, and cornea.

Currently, preserving solid organs depends on rapid intravascular cooling done in situ, followed by removal of the organs, storage of the organs in ice-cold preservation fluid and rapid transport to the recipients' hospitals. The cold ischemic time is the length of time the organs are on ice, without blood flow. The maximum cold ischemic time limits the amount of time that can pass between organ recovery and the organ transplant. Between 2%-10% of matched and procured organs cannot be used due to extended ischemic time, depending on the type of organ. Similarly, approximately 10 to 20% of procured organs are not used due to poor organ function and/or infection (not including HIV/CMV/hepatitis).

Current preservation techniques involve the use of ice-cold solutions that include electrolytes, antioxidants, hydrogen ion buffers and sugars (Punch et al., 2001). Appropriate tissue matching depends on blood group matching (e.g., blood type, A, B or O) for all organs. Immunosuppresive regimens typically include three drugs: a glucocorticoid such as prednisone, an antimetabolite such as azathiprine or mycophenolate, and a calcineurin inhibitor such as cyclosporine or tacrolimus.

The two most frequently used methods for preserving/transporting hearts for transplantation are hypothermic storage and continuous perfusion. In the former method, the heart is arrested, removed from the donor, and then rapidly cooled and transported in cold storage. In the latter method, the following steps are typically employed: 1) pulsatile flow; 2) hypothermia; 3) membrane oxygenation, and 4) a perfusate containing both.

The methods of the present invention may be used to increase the survivability of donor tissues and organs, thereby extending the time before the donor tissue must be transplanted into a recipient and blood flow restored. These methods may be combined with current preservation methods, including the use of preservation agents and oxygen perfusion. A variety of preservation solutions have been disclosed in which the organ is surrounded or perfused with the preservation solution while it is transported. One of the most commonly used solution is ViaSpan® (Belzer UW), which can be combined with cold storage. Other examples of such solutions or components of such solutions include the St. Thomas solution (Ledingham et al. 1987), Broussais solution, UW solution (Ledingham et al. 1990), Celsior solution (Menasche et al. 1994), Stanford University solution, and solution B2O (Bernard et al. 1985), as well as those described and/or claimed in U.S. Pat. Nos. 6,524,785; 6,492,103; 6,365,338; 6,054,261; 5,719,174; 5,693,462; 5,599,659; 5,552,267; 5,405,742; 5,370,989; 5,066,578; 4,938,961; and, 4,798,824. In addition to solutions, other types of materials are also known for use in transporting organs and tissue. These include gelatinous or other semi-solid material, such as those described, for example, in U.S. Pat. No. 5,736,397.

Some of the systems and solutions for organ preservation specifically involve oxygen perfusion in the solution or system to expose the organ to oxygen, because it is believed that maintaining the organ or tissue in an oxygenated environment improves viability. See Kuroda et al., (Transplantation 46(3):457-460, 1988) and U.S. Pat. Nos. 6,490,880; 6,046,046; 5,476,763; 5,285,657; 3,995,444; 3,881,990; and, 3,777,507. A variety of systems and containers for transporting organs and tissues have been developed, which provide cooling and/or oxygen perfusion. These may be employed in combination with contacting the tissue or organ with sulfide and nitric oxide according to the present invention. Specific apparatuses include, for example, cooling systems described in U.S. Pat. Nos. 4,292,817, 4,473,637, 4,745,759, 5,434,045 and 4,723,974. Others constitute a system in which an apparatus is devised for perfusion of the organ or tissue in a preservation solution, as is described in U.S. Pat. Nos. 6,490,880; 6,100,082; 6,046,046; 5,326,706; 5,285,657; 5,157,930; 4,951,482; 4,502,295; and, 4,186,565.

In certain embodiments, the present invention provides methods to enhance survivability of platelets. Platelets are small cell fragments (˜⅓ size of erythrocytes) that play a vital role in the formation of blood clots at the site of bleeding. Platelet concentrates are transfused for a variety of indications, for example: 1) to prevent bleeding due to thrombocytopenia; 2) in a bleeding patient to maintain a platelet count above 50,000; 3) to address abnormal platelet function that is congenital or due to medications, sepsis, malignancy, tissue trauma, obstetrical complications, extra corporeal circulation, or organ failure such as liver or kidney disease.

Each unit of platelets contains an average of 0.8-0.85×10¹¹ platelets. Platelet concentrates also contain about 60 mL of plasma (coagulation factors) and small numbers of red blood cells and leukocytes. Platelet units must be maintained at room temperature (20° C.-24° C.) and agitated during storage. They can be stored at the Blood Center for up to 5 days. Longer storage is not possible at present due to deterioration of the platelets, and the risk of microbial contamination. Two sources of platelets currently exist: (1) pooled random donor platelet concentrates prepared from platelets that have been harvested by centrifuging units of whole blood; and (2) apheresis platelets, collected from a single donor, prepared in standard (equivalent to −4 pooled units) and “large” (equivalent to ˜6 pooled units) sizes.

Platelet storage poses problems that are not found with the storage of whole blood or other components. While whole blood, red and white cells may be stored at 4° C. for weeks, platelets will aggregate in cold storage and when allowed to settle. Therefore, the standard method of storing platelets is at room temperature, approximately 20 to 24° C., with gentle agitation. Even under these conditions, platelets can only be stored for 5 days before they need to be discarded. This problem of outdating platelets results in approximately $500 million annually in lost revenue for US hospitals. If even a moderate increase in shelf life could be attained, approximately 90% of this loss could be avoided.

An additional problem with platelet storage is bacterial contamination. Contamination is primarily due to staphylococci from the skin during the phlebotomy, or else donor bacteremia. The bacterial contamination of platelets represents the largest infectious risk with any blood transfusion procedure.

A significant factor affecting the viability of platelets is regulation of pH. Virtually all units of platelets stored according to the currently accepted methods show a decrease in pH from their initial value of approximately 7.0. This decrease is primarily due to the production of lactic acid by platelet glycolysis and to a lesser extent to accumulation of CO₂ from oxidative phosphorylation. As the pH falls, the platelets change shape from discs to spheres. If the pH falls below 6.0, irreversible changes in platelet morphology and physiology render them non-viable after transfusion. An important goal in platelet preservation, therefore, is to prevent this decrease in pH. It was previously thought that platelets must be stored in a container permeable to oxygen since glycolysis is stimulated when oxygen availability is limited (see e.g., U.S. Pat. No. 5,569,579). However, it has more recently been demonstrated that the viability of stored platelets can be extended by storing them in an anoxic environment.

The present invention provides methods of enhancing survivability of platelets, including, in particular embodiments, platelets stored in an anoxic environment, comprising contacting the platelets with an effective amount of sulfide and nitric oxide during storage.

In various embodiments of the methods of the present invention, including those specifically exemplified above, biological material is exposed to sulfide and nitric oxide once or more than one time. In certain embodiments, biological matter is exposed to sulfide and nitric oxide 1, 2, 3, 4, 5, 6, 7, 8, 9, 10 or more times, meaning when a biological matter is exposed multiple times that there are periods of respite (with respect to exposure to the active compound) in between.

It is also contemplated that sulfide and nitric oxide may be administered before, during, after, or any combination thereof, in relation to the onset or progression of an injurious insult or disease condition. In certain embodiments, pre-treatment of biological matter with sulfide and nitric oxide is sufficient to enhance survivability and/or reduce damage from an injurious or disease insult. Pre-treatment is defined as exposure of the biological matter to sulfide and nitric oxide before the onset or detection of the injurious or disease insult. Pre-treatment can be followed by termination of exposure at or near the onset of the insult or continued exposure after the onset of insult.

In various embodiments, the present invention comprises contacting living biological matter with an effective amount of sulfide and nitric oxide. As previously noted, the term “effective amount” means an amount that can achieve the stated result. In certain methods of the present invention, an “effective amount” is, for example, an amount that enhances the survivability of biological matter in response to ischemic or hypoxic conditions, or an amount that protects biological material from injury due to ischemic or hypoxic conditions.

### B. Nitric Oxide and Sulfur Compositions and Formulations

The methods of the present invention may be practiced using a variety of different formulations of nitric oxide and sulfide, including both gas and liquid formulations of each, as well as gas and liquid coformulations comprising both nitric oxide and sulfide. In particular embodiments, any of the following formulations of nitric oxide or sulfide are used.

### 1. Nitric Oxide Formulations and Methods of Manufacture

Nitric oxide may be administered as either a gas or a liquid. In addition, nitric oxide may be directly administered or provided in the form of a prodrug, metabolite or analog, including prodrug forms that release nitric oxide (see U.S. Pat. No. 7,122,529). For instance, a nitric oxide producing compound, composition or substance may undergo a thermal, chemical, ultrasonic, electrochemical, metabolic or other reaction, or a combination of such reactions, to produce or provide nitric oxide, or to produce its chemical or biological effects. Thus, certain embodiments of the present invention include various nitric oxide and nitric oxide prodrugs, including any nitric oxide producing compound, composition or substance. Certain embodiments of the present invention are directed to nitric oxide precursors and catalysts, such as L-arginine, and analogs and derivatives thereof, and nitric oxide synthases (NOS), and mutants/variants thereof.

Various embodiments of the present invention are directed to nitric oxide donors or analogs, which generally donate nitric oxide or a related redox species and more generally provide nitric oxide bioactivity. Examples of nitric oxide donors or analogs include ethyl nitrite, diethylamine NONOate, diethylamine NONOate/AM, spermine NONOate, nitroglycerin, nitroprusside, NOC compounds, NOR compounds, organic nitrates (e.g., glycerin trinitrate), nitrites, furoxan derivatives, N-hydroxy (N-nitrosamine) and perfluorocarbons that have been saturated with NO or a hydrophobic NO donor.

Additional examples of nitric oxide donors or analogs include S-nitroso, O-nitroso, C-nitroso and N-nitroso compounds and nitro derivatives thereof, such as S-nitrosoglutathione, S-nitrosothiols, nitroso-N-acetylpenicillamine, S-nitroso-cysteine and ethyl ester thereof, S-nitroso cysteinyl glycine, S-nitroso-gamma-methyl-L-homocysteine, S-nitroso-L-homocysteine, S-nitroso-gamma-thio-L-leucine, S-nitroso-delta-thio-L-leucine, S-nitrosoalbumin, S-Nitroso-N-penicillamine (SNAP), glyco-SNAPs, fructose-SNAP-1. Further examples of nitric oxide donors or analogs include metal NO complexes, isosorbide mononitrate, isosorbide dinitrate, molsodomines such as Sin-1, streptozotocin, dephostatin, 1,3-(nitrooxymethyl)phenyl 2-hydroxybenzoate and related compounds (see U.S. Pat. No. 6,538,033); NO complexes with cardiovascular amines, such as angiopeptin, heparin, and hirudin, arginine, and peptides with an RGD sequence (See U.S. Pat. No. 5,482,925); diazeniumdiolates such as ionic diazeniumdiolates, O-derivatised diazeniumdiolates, C-based diazeniumdiolates, and polymer based diazeniumdiolates.

In certain embodiments, formulations of nitric oxide suitable for administration according to embodiments of the present invention are liquid solutions. Such solutions may comprise water, dextrose, or saline, polymer-bound compositions dissolved in diluents; other aqueous or nonaqueous solvents, such as vegetable oil, synthetic aliphatic acid glycerides, esters of higher aliphatic acids or propylene glycol, including the addition of conventional additives such as solubilizers, isotonic agents, suspending agents, emulsifying agents, stabilizers and preservatives; capsules, sachets or tablets, each containing a predetermined amount of the nitric oxide; solids or granules; suspensions in an appropriate liquid; suitable emulsions; and gases and/or aerosols, for example, as used in inhalation and nebulizer therapy (see, e.g, U.S. Pat. Nos. 5,823,180 and 6,314,956).

In particular embodiments, the present invention includes aerosol formulations, which may include aqueous solutions, lipid soluble aqueous solution, and micronized powders. In certain embodiments the aerosol particle size is between about 0.5 micrometers and about 10 micrometers. Aerosols may be generated by a nebulizer or any other appropriate means.

With respect to gas formulations, those compounds/compositions that are either normally gases or have been otherwise converted to gases may be formulated for use by dilution in nitrogen and/or other inert gases and may be administered in admixture with oxygen, hydrogen sulfide, air, and/or any other appropriate gas or combination of multiple gases at a desired ratio. Dilution, for example, to a concentration of 1 to 100 ppm is typically appropriate. In particular embodiments, nitric oxide is used in the range of 10-80 ppm mixed into air.

In one embodiment, nitric oxide and oxygen are generally administered to a patient by diluting a nitrogen-nitric oxide concentrate gas containing about 1000 ppm nitric oxide with oxygen or oxygen-enriched air carrier gas to produce an inhalation gas containing nitric oxide in the desired concentration range (usually about 0.5 to 200 ppm, based on the total volume of the inhalation gas) (see: U.S. Pat. No. 5,692,495).

Polymer-bound compounds/compositions of the present invention may also be used; such compositions are capable of releasing nitric oxide, donors, analogs, precursors, etc., in an aqueous solution and preferably release nitric oxide, etc., under physiological conditions. Any of a wide variety of polymers can be used in the context of the present invention. It is only necessary that the polymer selected is biologically acceptable. Illustrative of polymer suitable for use in the present invention include polyolefins, such as polystyrene, polypropylene, polyethylene, polytetrafluorethylene, polyvinylidene difluoride, and polyvinylchloride, polyethylenimine or derivatives thereof, polyethers such as polyethyleneglycol, polyesters such as poly(lactide/glycolide), polyamides such as nylon, polyurethanes, biopolymers such as peptides, proteins, oligonucleotides, antibodies and nucleic acids, starburst dendrimers, and the like.

The amount of the compounds/compositions of the present invention to be used as a therapeutic agent, of course, varies according to the compounds/compositions administered, the type of disorder or condition encountered and the route of administration chosen. A suitable dosage is thought to be about 0.01 to 10.0 mg/kg of body weight/day. The preferred dosage is, of course, that amount just sufficient to treat a particular disorder or condition and would preferably be an amount from about 0.05 to 5.0 mg/kg of body weight/day.

When either nitric oxide or sulfide are administered as gases, a suitable dosage is thought to be between 1 ppm (parts per million) and 1000 ppm, preferentially between 5 ppm and 200 ppm.

### 2. Sulfide Formulations and Methods of Manufacture

Sulfide may be administered as either a gas or a liquid. Accordingly, the present invention includes the administration of both gaseous and liquid formulations of sulfide or other sulfur-containing compound. A variety of gaseous formulations of sulfide are described, e.g., in U.S. patent application Ser. No. 11/408,734, and liquid compositions of sulfide are described in U.S. Provisional Patent Application No. 60/849,900. Any of these compounds and liquid compositions of sulfide may be used according to the present invention.

In particular embodiments, it is specifically contemplated that the sulfide that is provided is hydrogen sulfide. However, it is also contemplated that other sulfur containing compounds may be administered instead of hydrogen sulfide. These include, e.g., sodium sulfide, sodium thiomethoxide, cysteamine, sodium thiocyanate, cysteamine-S-phosphate sodium salt, or tetrahydrothiopryan-4-ol.

In certain embodiments, the pharmaceutical composition provides an effective dose of H₂S to provide when administered to a patient a Cₘₐₓ or a steady state plasma concentration of between 1 μM to 10 mM, between about 1 μM to about 1 mM, or between about 10 μM to about 500 μM. In relating dosing of hydrogen sulfide to dosing with sulfide salts, in typical embodiments, the dosing of the salt is based on administering approximately the same sulfur equivalents as the dosing of the H₂S. Appropriate measures will be taken to consider and evaluate levels of sulfur already in the blood.

A gaseous form or salt of H₂S is specifically contemplated in some aspects of the invention. With hydrogen sulfide gas, for example, in some embodiments, the concentration may be from about 0.01 to about 0.5 M (at STP). Typical levels of hydrogen sulfide contemplated for use in accordance with the present invention include values of about 1 to about 150 ppm, about 10 to about 140 ppm, about 20 to about 130 ppm, and about 40 to about 120 ppm, or the equivalent oral, intravenous or transdermal dosage thereof. Other relevant ranges include about 10 to about 80 ppm, about 20 to about 80 ppm, about 10 to about 70 ppm, about 20 to about 70 ppm, about 20 to about 60 ppm, and about 30 to about 60 ppm, or the equivalent oral, intravenous or transdermal thereof. It also is contemplated that, for a given animal in a given time period, the sulfide atmosphere should be reduced to avoid a potentially lethal build up of sulfide in the subject. For example, an initial environmental concentration of 80 ppm may be reduced after 30 min to 60 ppm, followed by further reductions at 1 hr (40 ppm) and 2 hrs (20 ppm).

In other embodiments, a liquid sulfide composition is contemplated. In certain embodiments, the concentration of the chalcogenide or salt or precursor thereof in a liquid chalcogenide composition of the present invention is about, at least about, or at most about 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0 mM or M or more or any range derivable therein (at standard temperature and pressure (STP)). In particular embodiments, liquid pharmaceutical compositions of the present invention comprise a sulfide wherein the concentration of sulfide is in the range 1 mM-250 mM. In another embodiment, the concentration of sulfide is in the range 10 mM-200 mM.

Liquid pharmaceutical compositions of the present invention may include a sulfur containing compound or salt or precursor thereof in any desired concentration. The concentration may be readily optimized, e.g., depending upon the type of biological matter being treated and the route of administration, so as to deliver an effective amount in a convenient manner and over an appropriate time-frame. In some embodiments, the concentration of sulfur-containing compound or salt or precursor thereof is in the range of 0.001 mM to 5,000 mM, in the range of 1 mM to 1000 mM, in the range of 50 to 500 mM, in the range of 75 to 250 mM, or in the range of 95 mM to 150 mM.

In one embodiment, the pH of a liquid pharmaceutical composition of the present invention is in the range of (5.0-9.0). The pH of the liquid pharmaceutical composition may be adjusted to a physiologically compatible range. For example, in one embodiment, the pH of the liquid pharmaceutical composition is in the range of 6.0-8.5. In another embodiment, the liquid pharmaceutical compositions of the present invention have a pH in the range of 7.0-8.0.

In one embodiment, methods of preparing liquid pharmaceutical compositions of the present invention further comprise adjusting the osmolarity of the liquid pharmaceutical composition to an osmolarity in the range of 200-400 mOsmol/L. In one embodiment, the osmolarity of the liquid pharmaceutical composition is in the range of 240-360 mOsmol/L or an isotonic range. In one embodiment, the osmolarity of the liquid pharmaceutical composition is in the range of 250-330 mOsmol/L.

In certain embodiments, isotonicity of liquid pharmaceutical compositions is desirable as it results in reduced pain upon administration and minimizes potential hemolytic effects associated with hypertonic or hypotonic compositions.

### 3. Coformulations of Nitric Oxide and Sulfide and Methods of Manufacture

The present invention further provides both gas and liquid compositions comprising both nitric oxide and sulfide.

a. Gas Coformulations

In one embodiment, the present invention provides a gas coformulation comprising gas nitric oxide and gas sulfide. In particular embodiments, the gas coformulation further comprises air.

In one embodiment, the amount of nitric oxide is about the same or exceeds any amount of hydrogen sulfide in the gas mixture. In one embodiment, the atmosphere will be close to 100% NO, but as will be evident to one skilled in the art, the amount of NO may be balanced with hydrogen sulfide gas and/or air. In this context, the ratio of nitric oxide to hydrogen sulfide is preferably 85:15 or greater, 199:1 or greater or 399:1 or greater. In another embodiment, the amount of sulfide is about the same or exceeds any amount of nitric oxide in the gas mixture. In one embodiment, the atmosphere will be close to 100% sulfide, but as will be evident to one skilled in the art, the amount of sulfide may be balanced with nitric oxide gas and/or air. In this context, the ratio of hydrogen sulfide to nitric oxide is preferably 85:15 or greater, 199:1 or greater or 399:1 or greater.

In certain embodiments, the ratio of either sulfide to nitric oxide or nitric oxide to sulfide is about, at least about, or at most about 1:1, 2:1, 2.5:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 110:1, 120:1, 130:1, 140:1, 150:1, 160:1, 170:1, 180:1, 190:1, 200:1, 210:1, 220:1, 230:1, 240:1, 250:1, 260:1, 270:1, 280:1, 290:1, 300:1, 310:1, 320:1, 330:1, 340:1, 350:1, 360:1, 370:1, 380:1, 390:1, 400:1, 410:1, 420:1, 430:1, 440:1, 450:1, 460:1, 470:1, 480:1, 490:1, 500:1 or more, or any range derivable therein.

In some cases, the amount of nitric oxide or sulfide is relative to each other, while in others, one or both are provided as absolute amounts. For example, in some embodiments of the invention, the amount of nitric oxide or sulfide is in terms of “parts per million (ppm),” which is a measure of the parts in volume of nitric oxide or sulfide, respectively, in a million parts of air at standard temperature and pressure of 20° C. and one atmosphere pressure. In one embodiment, the balance of the gas volume is made up with hydrogen sulfide or nitric oxide, respectively. In one embodiment, nitric oxide is included at an effective concentration, and the balance of the gas volume is made up with hydrogen sulfide. Alternatively, the balance of the gas volume may include sulfide at an effective amount and remainder as air. In another embodiment, sulfide is included at an effective concentration, and the balance of the gas volume is made up with nitric oxide. In another embodiment, the balance of the gas volume may include nitric oxide at an effective amount and remainder as air. In specific embodiments, a gas composition includes nitric oxide at a concentration of 1-150 or 10-80 ppm and sulfide at a concentration of 1-150 or 10-80 ppm, with the remainder of the gas volume made up with air. In one embodiment, the amount of nitric oxide to hydrogen sulfide is related in terms of parts per million of nitric oxide balanced with hydrogen sulfide.

In particular embodiments, it is contemplated that the atmosphere to which the biological material is exposed or incubated may be at least 0, 10, 20, 40, 60, 80, 100, or 200, parts per million (ppm) of nitric oxide balanced with hydrogen sulfide and in some cases sulfide mixed with a non-toxic and/or non-reactive gas and/or air

In one embodiment, co-administration of NO and sulfide to biological matter, comprises nitric oxide and sulfide gases formulated separately in pressurized gas cylinders wherein a known concentration of NO or sulfide is mixed with an inert gas (e.g., nitrogen or argon), wherein the ratio of NO to sulfide can be adjusted by mixing of the container contents at various flow rates prior to exposing the biological matter to the mixture of NO and sulfide. The ratio of NO and sulfide may be varied.

In one embodiment, co-administration of NO and sulfide to biological matter, comprises nitric oxide and sulfide gases formulated together in a single pressurized gas cylinder wherein known concentrations of both NO and sulfide are mixed with an inert gas (e.g., nitrogen or argon) and the ratio of NO to sulfide is fixed.

In either embodiment, it is contemplated that the NO/sulfide mixture is further mixed with air or oxygen prior to exposure to the biological matter. Devices that can monitor the absolute concentrations of NO and sulfide and that can blend NO, sulfide, air and oxygen in defined concentrations are known to those skilled in the art and further described herein.

Alternatively, the atmosphere may be expressed in terms of kPa. It is generally understood that 1 million parts=101 kPa at 1 atmosphere. In embodiments of the invention, the environment in which a biological material is incubated or exposed to is about, at least about, or at most about 0.001, 0.005, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.70, 0.75, 0.80, 0.5, 0.90, 0.95, 1.0 kPa or more nitric oxide, or any range derivable therein. As described above, such levels can be balanced with hydrogen sulfide and/or other non-toxic and/or non-reactive gas(es). Also, the atmosphere may be defined in terms of NO levels in kPa units. In certain embodiments, the atmosphere is about, at least about, or at most about 1, 5, 10, 15, 20, 25, 30, 35, 40, 45, 50, 55, 60, 65, 70, 75, 80, 85, 90, 95, 101, 101.3 kPa NO, or any range derivable therein. In particular embodiments, the partial pressure is about or at least about 85, 90, 95, 101, 101.3 kPa NO, or any range derivable therein.

In embodiments of the invention, the environment in which a biological material is incubated or exposed to is about, at least about, or at most about 0.001, 0.005, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.35, 0.40, 0.45, 0.50, 0.55, 0.60, 0.65, 0.70, 0.75, 0.80, 0.5, 0.90, 0.95, 1.0 kPa or more sulfide, or any range derivable therein. As described above, such levels can be balanced with nitric oxide and/or other non-toxic and/or non-reactive gas(es). Also, the atmosphere may be defined in terms of sulfide levels in kPa units. In certain embodiments, the atmosphere is about, at least about, or at most about 1, 5, 10, 15, 20, 25, 30, 35, 40, 45, 50, 55, 60, 65, 70, 75, 80, 85, 90, 95, 101, 101.3 kPa sulfide, or any range derivable therein. In particular embodiments, the partial pressure is about or at least about 85, 90, 95, 101, 101.3 kPa sulfide, or any range derivable therein.

b. Liquid Coformulations

The present invention provides liquid formulations or compositions comprising both sulfide and nitric oxide. The present invention also provides methods of preparing such formulations, as demonstrated in the Examples. In certain embodiments, liquid formulations of sulfide are prepared essentially as described in U.S. Provisional Patent Application No. 60/849,900, and nitric oxide is added to the resulting formulation, e.g., by bubbling nitric oxide gas into the sulfide liquid formulation.

Liquid pharmaceutical compositions of the present invention may include sulfide in any desired concentration. In particular embodiments, the concentration of sulfide is optimized to be therapeutically effective for its intended purpose. In another embodiment, the concentration of sulfide is optimized to be effective in reducing the undesired side-effects of nitric oxide. The concentration may be readily optimized, e.g., depending upon the type of biological matter being treated and the route of administration, so as to deliver an effective amount in a convenient manner and over an appropriate time-frame. In some embodiments, the concentration of sulfide or salt or precursor thereof is in the range of 0.001 mM to 5,000 mM, in the range of 1 mM to 1000 mM, in the range of 50 to 500 mM, in the range of 75 to 250 mM, or in the range of 95 mM to 150 mM. The liquid pharmaceutical compositions of the present invention further comprise sulfide wherein the concentration of sulfide is in the range 1 mM-250 mM. In another embodiment, the concentration of sulfide is in the range 10 mM-200 mM.

Liquid pharmaceutical compositions of the present invention may include nitric oxide in any desired concentration. In particular embodiments, the concentration of nitric oxide is optimized to be therapeutically effective for its intended purpose. In another embodiment, the concentration of nitric oxide is optimized to be effective in reducing the undesired side-effects of sulfide. The concentration may be readily optimized, e.g., depending upon the type of biological matter being treated and the route of administration, so as to deliver an effective amount in a convenient manner and over an appropriate time-frame. In one embodiment, the concentration of nitric oxide is in the range of 1 μM-3 mM in the pharmaceutical composition. In one embodiment, the concentration of nitric oxide is in the range of 10 μM-2 mM in the pharmaceutical composition. In one particular embodiment, the concentration of nitric oxide is in the range of 100 μM-2 mM in the pharmaceutical composition.

In various embodiments, the liquid composition is prepared in a liquid or solution in which the oxygen has been reduced prior to contacting the liquid or solution with nitric oxide or sulfide. Examples of suitable liquids include water and phosphate-buffered saline. Particular embodiments of the present invention further comprise limiting oxygen content in each aspect of manufacturing and storage of the pharmaceutical composition. In one embodiment, oxygen is measured in the range of 0 μM-5 μM in the pharmaceutical composition. In one embodiment, oxygen is measured in the range of 0 μM-3 μM in the pharmaceutical composition. In one embodiment, oxygen is measured in the range of 0.001 μM-0.1 μM in the pharmaceutical composition. In one embodiment, oxygen is measured in the range of 0.1 μM-1 μM in the pharmaceutical composition.

Nitric oxide and sulfide are not stable in the presence of oxygen due to their ability to react chemically with oxygen, leading to their oxidation and chemical transformation. Accordingly, oxygen may be removed from liquids or solutions using methods known in the art, including, but not limited to, application of negative pressure (vacuum degasing) to the liquid or solution, or contacting the solution or liquid with a reagent which causes oxygen to be bound or “chelated”, effectively removing it from solution. In particular embodiments, oxygen is removed from the coformulations of the present invention.

In one embodiment, a stock solution of sulfide (e.g., 2.5M) is prepared by dissolving Na₂S.9H₂O crystals in deoxygenated water. The stock solution is then diluted into deoxygenated water to produce a Na₂S solution (e.g., 200 mM). Nitric oxide is then bubbled into the Na₂S solution in an oxygen-free environment. The resulting coformulation may then be pH adjusted to a final pH of 7.0-8.0.

In another embodiment, aqueous nitric oxide is prepared by saturating pure NO gas and hydrolyzing 1 mM 1-hydroxy-2-oxo-3(N-methyl-3-aminoethyl)-3-methyl-1-triazene (NOC-7) in an oxygen-free environment using a modified Saltzman method, essentially as described in Ohkawa et al, Nitric Oxide (2001) 5:515). A solution of aqueous sulfide is prepared by dissolving Na₂S.9H₂O crystals in deoxygenated water (e.g., 200 mM). The aqueous nitric oxide composition is then combined with the aqueous sulfide composition to produce a liquid composition comprising both nitric oxide and sulfide. The pH may be adjusted to a final pH of 7.0-8.0, if desired.

In another embodiment, aqueous nitric oxide is prepared by saturating pure NO gas and hydrolyzing 1 mM 1-hydroxy-2-oxo-3(N-methyl-3-aminoehtyl)-3-methyl-1-triazene (NOC-7) in an oxygen-free environment using a modified Saltzman method, essentially as described in Ohkawa et al., Nitric Oxide (2001) 5:515). Hydrogen sulfide gas is then bubbled into the nitric oxide solution. The pH may be adjusted to a final pH of 7.0-8.0, as desired.

In certain embodiments, the liquid formulations are manufactured in a sealed container that contains a vessel to hold the liquid pharmaceutical composition with access ports for pH measurement, addition of gasses, and dispensing without contact to the outside atmosphere. In one embodiment, the vessel is a three neck flask with ground glass fittings. In one embodiment, the vessel is flushed with nitrogen gas or argon gas to minimize oxygen content to a range of 0.00 μM-3 μM.

In certain embodiments, the solution is dispensed from the flask under positive argon pressure into vials or bottles by filling the headspace with argon to the maximum to prevent oxygen to enter the solution. The dispensing vials or bottles are placed in a glove box that is flushed with a constant stream of argon to minimize oxygen to a range of 0.00 μM-0.5 μM and each bottle or vial is flushed with argon before dispensing. The vials and bottles are made of amber glass to enhance stability and are closed with caps lined with Teflon lined silicon or rubber sealed with plastic caps and using a crown-cap crimper to provide an air-tight seal. In one embodiment, the vials and bottles are comprised of borosilicate glass. In one embodiment, the vials and bottles are comprised of silicon dioxide.

In one embodiment, the liquid pharmaceutical composition is stored in an impermeable container. This is particularly desirable when the oxygen has previously been removed from the solution to limit or prevent oxidation of the pharmaceutical or salt or precursor thereof. Additionally, storage in an impermeable container will inhibit the oxidation products of the pharmaceutical gas from the liquid or solution, allowing a constant concentration of the dissolved pharmaceutical to be maintained. Impermeable containers are known to those skilled in the art and include, but are not limited to, “i.v. bags” comprising a gas impermeable construction material, or a sealed glass vial. To prevent exposure to air in the gas-tight storage container, an inert or noble gas, such as nitrogen or argon, may be introduced into the container prior to closure.

In other related embodiments, liquid pharmaceutical compositions are stored in a light-resistant or a light-protective container or vial, such as an amber vial. The composition is preferably packaged in a glass vial. It is preferably filled to a slight over-pressure in an inert atmosphere, e.g., nitrogen, to prevent/slow oxidative breakdown of the composition, and is contained in a form such that ingress of light is prevented, thereby preventing photochemical degradation of the composition. This may be most effectively achieved using an amber vial. Container systems that permit a solution to be stored in an oxygen-free environment are well known as many intravenous solutions are sensitive to oxygen. For example, a glass container that is purged of oxygen during the filling and sealing process may be used. In another embodiment, flexible plastic containers are available that may be enclosed in an overwrap to seal against oxygen. Basically, any container that prevents oxygen from interacting with the liquid pharmaceutical composition may be used. (see: U.S. Pat. No. 6,458,758) In one embodiment, the container includes one or more oxygen scavenger. For example, the oxygen scavenging composition can be applied as a coating or lining upon the inside surface of the product supporting or retaining means to function as a barrier to oxygen permeation (see: U.S. Pat. No. 5,492,742).

### 4. Nitric Oxide and Sulfur Products

The pharmaceutical compositions of the present invention may comprise one or more nitric oxide and/or sulfur products. In various embodiments, one or more nitric oxide or sulfur products is present in an amount less than 20%, less than 10%, less than 6.0%, less than 3.0%, less than 1.0%, less than 0.5%, less than 0.2%, less than 0.1%, less than 0.05%, or less than 0.01%. As used herein, the term “%” when used without qualification (as with w/v, v/v, or w/w) means % weight-in-volume for solutions of solids in liquids (w/v), % weight-in-volume for solutions of gases in liquids (w/v), % volume-in-volume for solutions of liquids in liquids (v/v) and weight-in-weight for mixtures of solids and semisolids (w/w) (Remington's Pharmaceutical Sciences (2005); 21^{st} Edition, Troy, David B. Ed. Lippincott, Williams and Wilkins).

In one embodiment, a nitric oxide product is a nitrosothiol. In one embodiment, the nitrosothiol product is present in the range of 0%-20% (w/v). In one embodiment, the nitrosothiol product is in the range of 4.0%-10.0% (w/v). In one embodiment, the nitrosothiol product is in the range of 3.0%-6.0% (w/v). In one embodiment the nitrosothiol product is in the range of 1.0%-3.0% (w/v). In one embodiment, the nitrosothiol product is in the range of 0%-1.0% (w/v).

In one embodiment, the peroxynitrite product is present in the range range of 4.0%-10.0% (w/v). In one embodiment, the nitrosothiol product is in the range of 3.0%-6.0% (w/v). In one embodiment the nitrosothiol product is in the range of 1.0%-3.0% (w/v). In one embodiment, the nitrosothiol product is in the range of 0%-1.0% (w/v).

The pharmaceutical composition of the present invention may further comprise sulfide oxidation products. Oxidation products of the present invention include, but are not limited to, sulfite, sulfate, thiosulfate, polysulfides, dithionate, polythionate, and elemental sulfur. In various embodiments, one or more of these oxidation products is present in an amount less than 10%, less than 6.0%, less than 3.0%, less than 1.0%, less than 0.5%, less than 0.2%, less than 0.1%, less than 0.05%, or less than 0.01%.

In one embodiment, the oxidation product, sulfite, is present in the range of 0%-10% (w/v). In one embodiment, the oxidation product, sulfite, is in the range of 3.0%-6.0% (w/v). In one embodiment the oxidation product, sulfite, is in the range of 1.0%-3.0% (w/v). In one embodiment, the oxidation product, sulfite, is in the range of 0%-1.0% (w/v).

In one embodiment, the oxidation product, sulfate, is present in the range of 0%-10.0% (w/v). In one embodiment, the oxidation product, sulfate, is in the range of 3.0%-6.0%(w/v). In one embodiment, the oxidation product, sulfate, is in the range of 1% to 3.0% (w/v). In one embodiment, the oxidation product, sulfate, is in the range of 0%-1.0% (w/v).

In one embodiment, the oxidation product, thiosulfate, is present in the range of 0%-10% (w/v). In another embodiment, the oxidation product, thiosulfate, is in the range of 3.0%-6.0% (w/v). In another embodiment, the oxidation product, thiosulfate, is in the range of 1.0%-3.0% (w/v). In another embodiment, the oxidation product, thiosulfate, is in the range of 0%-1.0% (w/v).

In one embodiment, the oxidation products include polysulfides present in the range of(0%-10% (w/v). In one embodiment, the oxidation products, polysulfides, are in the range of 3.0%-6.0% (w/v). In one embodiment the oxidation products, polysulfides, are in the range of 1.0%-3.0% (w/v). In one embodiment, the oxidation products, polysulfides, are in the range of 0%-1.0% (w/v).

In one embodiment, the oxidation product, dithionate, is present in the range of 0%-10% (w/v). In one embodiment, the oxidation product, dithionate, is in the range of 3.0%-6.0% (w/v). In one embodiment the oxidation product, dithionate, is in the range of 1.0%-3.0% (w/v). In one embodiment, the oxidation product, dithionate, in the range of 0%-1.0% (w/v).

In one embodiment, the oxidation product, polythionate, is present in the range of 0%-10% (w/v). In one embodiment, the oxidation product, polythionate, is in the range of 3.0%-6.0% (w/v). In one embodiment the oxidation product, polythionate, is in the range of 1.0%-3.0% (w/v). In one embodiment, the oxidation product, polythionate, is in the range of 0%-1.0% (w/v).

In one embodiment, the oxidation product, elemental sulfur, is present in the range of 0%-10% (w/v). In one embodiment, the oxidation product, elemental sulfur, is in the range of 3.0%-6.0% (w/v). In one embodiment the oxidation product, elemental sulfur, is in the range of 1.0%-3.0% (w/v). In one embodiment, the oxidation product, elemental sulfur, is present in the range of 0%-1.0% (w/v).

### 5. Pharmaceutical Compositions and Routes of Delivery

The present invention contemplates the administration of gas and liquid compositions described herein to patients, including humans and other mammals. Therefore, the present invention includes all pharmaceutical compositions comprising either or both nitric oxide and sulfide.

In some embodiments, compositions of the present invention are pharmaceutically acceptable parenteral formulations (e.g., intravenous, intra-arterial, subcutaneous, intramuscular, intracisternal, intraperitoneal, and intradermal) dosage forms. In other embodiments, liquid pharmaceutical compositions are formulated for oral, nasal (inhalation or aerosol), nebulizer, buccal, or topical administration dosage forms.

In various embodiments, methods of the present invention include deliver by any suitable route. Accordingly, in certain embodiments, methods of the invention include and compositions of the present invention may be administered through inhalation, injection, catheterization, immersion, lavage, perfusion, topical application, absorption, adsorption, or oral administration. intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, or via a lavage.

The parenteral liquid compositions may be buffered to a certain pH to enhance the solubility of the nitric oxide and/or sulfide or to influence the ionization state of the nitric oxide and/or sulfide. In addition, the compositions described herein may further include the addition of one or more of a metal chelator, a free radical scavenger, and/or a reducing agent.

The compositions and formulations of the present invention are, in certain embodiments, formulated for pharmaceutical use. Accordingly, they may include a variety of different pharmaceutical excipients and carriers, and may be formulated for pharmaceutical use as described, e.g., in U.S. Provisional Application No. 60/868,778.

The effective concentration of nitric oxide gas to achieve a therapeutic effect in a human depends on the dosage form and route of administration. For inhalation, in some embodiments effective concentrations are in the range of 5 ppm to 100 ppm, delivered intermittently or continuously. The effective concentration of liquid nitric oxide formulations is in the range of 0.01 mg/kg to 100 mg/kg, preferably 0.1 mg/kg to 10 mg/kg, delivered continuously or intermittently.

The effective concentration of hydrogen sulfide to achieve a therapeutic effect in a human depends on the dosage form and route of administration. For inhalation, in some embodiments, effective concentrations are in the range of 10 ppm to 500 ppm, delivered intermittently or continuously. The effective concentration of liquid sulfide formulations are in the range of 0.01 mg/kg to 100 mg/kg, preferably 0.1 mg/kg to 10 mg/kg, delivered continuously or intermittently.

The effective concentration of hydrogen sulfide to achieve stasis in a human depends on the dosage form and route of administration. For inhalation, in some embodiments, effective concentrations are in the range of 50 ppm to 500 ppm, delivered intermittently or continuously.

### C. Devices and Kits for the Preparation and Administration of Combinations of Nitric Oxide and Sulfide

In certain embodiments, methods of the invention are practiced using a specific delivery device or apparatus. Any method discussed herein can be implemented with any device for delivery or administration including, but not limited to, those discussed herein or described in PCT application WO/2006/113914. In one embodiment, hydrogen sulfide gas or nitric oxide gas or hydrogen sulfide gas and nitric oxide gas may be administered and levels monitored by gas delivery systems well known in the art (see, e.g., U.S. Pat. No. 6,109,260; U.S. Pat. No. 6,581,592; U.S. Pat. No. 6,089,229; U.S. Pat. No. 6,125,846; U.S. Pat. No. 5,839,433; U.S. Pat. No. 5,692,495; U.S. Pat. No. 6,164,276; U.S. Pat. No. 5,732,693; U.S. Pat. No. 5,558,083). It is contemplated that either hydrogen sulfide gas or nitric oxide gas or hydrogen sulfide gas and nitric oxide gas may be administered by the gas delivery devices described herein.

In certain embodiments, gas delivery devices described in US 2005/013625, US 2005/0147692, or US 2005/0170019 may be used to administer gas to a cell, tissue organ, organ system or organism. In one embodiment, gases may be administered using an implantable medical device for controlled release of gaseous agents (see: U.S. Pat. No. 7,122,027).

Additional exemplary devices include electrohydrodynamic (EHD) aerosol delivery devices and EHD aerosol devices use electrical energy to aerosolize liquid drag solutions or suspensions (see e.g., Noakes et al., U.S. Pat. No. 4,765,539; Coffee, U.S. Pat. No. 4,962,885; Coffee, PCT Application, WO 94/12285; Coffee, PCT Application, WO 94/14543; Coffee, PCT Application, WO 95/26234, Coffee, PCT Application, WO 95/26235, Coffee, PCT Application, WO 95/32807. EHD aerosol devices may more efficiently deliver drags to the lung than existing pulmonary delivery technologies.

In certain embodiments, methods of the present invention are practiced using a nebulizer. Nebulizers create aerosols from liquid drag formulations by using, for example, ultrasonic energy to form fine particles that may be readily inhaled. Examples of nebulizers include devices supplied by Sheffield/Systemic Pulmonary Delivery Ltd. (See, Armer et al, U.S. Pat. No. 5,954,047; van der Linden et al, U.S. Pat. No. 5,950,619; van der Linden et al., U.S. Pat. No. 5,970,974), Intal nebulizer solution by Aventis, (e.g., world wide web at fda.gov/medwatch/SAFETY/2004/feb_PI/Intal_Nebulizer_Pl.pdf).

For administration of a gas directly to the lungs by inhalation, various delivery methods currently available in the market for delivering oxygen may be used. For example, a resuscitator such as an ambu-bag may be employed (see U.S. Pat Nos. 5,988,162 and 4,790,327). An ambu-bag consists of a flexible squeeze bag attached to a face mask, which is used by the physician to introduce air/gas into the casualty's lungs. A portable, handheld medicine delivery device capable producing atomized agents that are adapted to be inhaled through a nebulizer by a patient suffering from a respiratory condition. In addition, such delivery device provides a means wherein the dose of the inhaled agent can be remotely monitored and, if required altered, by a physician or doctor. See U.S. Pat. No. 7,013,894. Delivery of the compound of invention may be accomplished by a method for the delivery of supplemental gas to a person combined with the monitoring of the ventilation of the person with both being accomplished without the use of a sealed face mask such as described in U.S. Pat No. 6,938,619. All the devices described here may have an exhaust system to bind or neutralize the compound of invention.

In one embodiment, the present invention includes a device for the metered coadministration of nitric oxide and sulfide to a patient, comprising a first compartment containing nitric oxide gas, a second compartment containing sulfide gas, wherein said first and second compartments are attached to a device for mixing the contained nitric oxide and sulfide gas prior to administration to a patient.

In another embodiment, the present invention includes a device for the metered coadministration of nitric oxide and sulfide to a patient, characterized by a gas feed system including a first line feeding nitric oxide, a second line feeding sulfide, a shut-off valve in the first line, a shut-off valve in the second line, wherein the first and second lines are in flow communication with a third line, whereby upon opening both shut-off valves to open flow nitric oxide and sulfide may flow through the first and second lines and into the third line, where they are mixed, and a device for delivering the resulting mixture of nitric oxide and sulfide to the patient, wherein said device is in flow communication with the third line. In particular embodiments, the device further include a fourth line feeding air and a shut-off valve in the fourth line, wherein the fourth line is in flow communication with the third line, whereby upon opening all shut-off valves to open flow nitric oxide, sulfide, and air may flow through the first, second, and third lines and into the third line, where they are mixed.

### Example 1

### Cytotoxic Effects of Nitric Oxide are Reduced by Treatment with Sulfide

The ability of a liquid pharmaceutical composition of hydrogen sulfide (liquid sulfide) to provide protective effects and reduce the cytotoxic effects of nitric oxide (NO) was tested in Murine J774 macrophages. The free radicals nitric oxide (NO) and superoxide (O2-) can result in rapid formation of peroxynitrite (ONOO—), a reactive cytotoxic oxidant species that is injurious to cells. In this study, it was shown that treatment with liquid sulfide produced cytoprotective benefits and reduced toxicity induced by nitric oxide byproducts, s-nitrosoglutathione (GSNO) and peroxynitrite (ONOO—).

Cell Culture and Treatment

Cells were cultured in 96-well plates until cells reached confluence essentially as described in C. Szabo and A. Salzman, Biochem and Biophys Res Comm. (1995) 209:739.

Cell Viability Measurements

Cell respiration, an indicator of cell viability, was assessed by the mitochondrial-dependent reduction of MTT to formazen (Gross and Levi, 1992). Cells in 96-well plates were incubated at 37° C. with MTT (0.2 mg/ml for 60 min). Culture medium was removed by aspiration, and the cells were solubilized in DMSO. The extent of reduction of MTT to formazan within cells was quantitated by OD₅₅₀ measurement.

GSNO Toxicity

H₂S was tested on cells to define the highest tested concentration that would reduce viability. It was determined that a concentration that may confer a protective effect was 1 mM. Cells were pretreated with liquid sulfide (either 100 μM or 1 mM) for 24 hours or left untreated. Following pretreatment, cells were treated with GSNO (1 mM, 3 mM, or 10 mM). Cell viability was measured at three hours.

ONOO— Toxicity

Cells were pretreated with liquid sulfide (60 μM) for 30 minutes or left untreated. Following pretreatment, ONOO— was added at concentrations of 0.3 mM, 0.6 mM, or 1 mM. Cell viability was measured at three hours.

Results

Cells were treated with various concentrations of liquid sulfide followed by incubation with either s-nitrosoglutathione (GSNO) or peroxynitrite (ONOO—). Three hours after treatment, cell viability was measured. As shown in FIG. 1, pretreatment of the cells with liquid sulfide reduced the cytotoxic effects of both GSNO (FIG. 1A) and ONOO— (FIG. 1B), demonstrating that sulfide can inhibit the toxic effects of NO or its byproducts (such as peroxynitrite).

In a related experiment, Murine J774 macrophages were grown until confluency in 96 well plates. Cells were pretreated with H₂S (30 mM, 60 mM, 100 mM, 1 mM) for 30 minutes or 24 hours, then cells were incubated with GSNO or ONOO— for 3 hours. GSNO was used in 1 mM and 3 mM concentrations, while ONOO— was used in 300 mM concentration. Following treatment, cells were incubated for 20 minutes with fresh media containing 0.05% MU. Media was discarded and replaced with 100 ml DMSO. Optical density was measured at 550 nm.

As shown in FIG. 2, pretreatment with H₂S for 30 minutes or 24 hours modulated S-nitroso-glutathione (GSNO) and peroxynitrite-induced alterations in J774 murine macrophages (FIG. 2A). 24 hours H₂S pretreatment significantly improves cell viability during GSNO and ONOO— treatment (FIG. 2B).

These results demonstrate that sulfide exerts acute and delayed cytoprotective effects in cultured macrophages. These effects are likely mediated by a direct antioxidant effect and a long-term “preconditioning” effect, respectively. They further demonstrate that sulfide may be useful in protecting cells from a variety of damaging agents, including free radicals and reactive oxygen species.

### Example 2

### Hydrogen Sulfide has Potent Anti-Inflammatory Effects in Vivo

An animal model was use to demonstrate that sulfide has anti-inflammatory effects in vivo. Four groups of C57/BI6 mice were subjected to bacterial lipopolysaccharide (5 mg/kg ip). Three groups received H₂S treatment (0.2 mg/kg/hr, 4 hrs), and a control group received saline using Alzet osmotic minipumps 30 minutes prior to the induction of endotoxaemia (n=7-10 /group) in both cases. The effect of the heme oxygenase inhibitor tin-protoporphyrin IX (6 mg/kg, ip, 30 min earlier to Alzet treatment) was also examined in two groups. After 4 hours, the animals were anesthetized using pentobarbital (60 mg/kg ip) and blood samples were taken. IL-1β and TNFα plasma levels were measured using a commercially available ELISA kit (R&D Systems).

As shown in FIG. 4, 30 min H₂S pretreatment significantly reduces LPS-induced IL-1 and TNF production in mice in vivo. The effect of H₂S on IL-1, but not on TNF was attenuated by pretreatment of the animals with Tin-protoporphyrin IX. The results demonstrate that H₂S exerts an anti-inflammatory effect in a murine model of inflammation, reducing the production of both IL-1 and TNF. In addition, it appears that some of this effect may be mediated by heme oxygenase.

### Example 3

### Preparation of Pharmaceutical Compositions Comprising Nitric Oxide and Hydrogen Sulfide

Liquid pharmaceutical compositions of the present invention are prepared according to the methods described herein.

Method of Manufacture

In one embodiment, liquid pharmaceutical compositions will be prepared in a fume hood in a basic glove box filled with nitrogen gas to yield an oxygen-free environment. The reactor with pH meter, bubbler and stirrer will be in the glove box. Oxygen levels in the glove box will be monitored with an oxygen meter (Mettler-Toledo) with a sensitivity level of 0.03 μM. Methods of preparing the liquid pharmaceutical compositions of the present invention include limiting oxygen content in each aspect of manufacturing and storage of the pharmaceutical composition where oxygen is measured in the range of 0 μM-5 μM in the pharmaceutical composition.

Liquid pharmaceutical compositions will be prepared in a three-neck flask (Wilmad Labs) with each opening fitted with ground glass fittings having the following features:
a) A universal adapter with a plastic cap with a central orifice and o-ring. This adapter will be fitted with a pH probe and sealed by the O-ring.
b) Universal adapter with a hose connector and a plastic cap with a central orifice and O-ring. This adapter will be fitted with a gas dispersion tube with a glass frit. The dispersion tube will be connected to a compressed gas cylinder and used to deoxygenate the solution by dissolving with compressed nitrogen and to neutralize the pH with a mixture of either nitric oxide, H₂S and nitrogen. The hose connector will be fitted with a plastic tube to allow pressure to escape. These two connections will be reversed to dispense the contents of the flask under positive nitrogen pressure.
c) The third neck will be sealed with a ground glass stopper and used to add Na₂S solution or water to the flask.

Dispensing and Storage

Liquid pharmaceutical compositions will be dispensed within the sealed Glove box, from the three-necked flask under positive nitrogen pressure. Amber vials or amber bottles will be filled to a slight over-pressure in an inert atmosphere argon or nitrogen to prevent/slow oxidative breakdown of the liquid pharmaceutical compositions, and sealed with plastic caps with Teflon/silicon liners or plastic caps with central Teflon lined silicon septa using a crown-cap crimper (Aldrich Z112976) to provide an air-tight seal.

### Composition 1: Hydrogen Sulfide Liquid and Nitric Oxide Gas

In this prophetic example, the novel composition will comprise a combination of nitric oxide gas and hydrogen sulfide liquid and will be prepared as follows. pH of 7.0 to 8.0 is required to maintain a sulfide concentration in the composition.

### Starting Materials

Nitric oxide qas: Various methods for the manufacture of nitric oxide for pharmaceutical administration exist. One process for the manufacture of nitric oxide results in the production of a gaseous nitric oxide product which contains little or no nitrous oxide (see: U.S. Pat. No. 5,670,127).

H₂S Liquid composition: Stock solutions will be prepared using deoxygenated water. The water will be deoxygenated by removing air under vacuum and dissolving with compressed nitrogen (99.99%) for 30 minutes. A saturated stock solution of 2.5 M Na₂S will be prepared from Na₂S.9H2O crystals (Fisher #5425) that will be rinsed with oxygen-free, distilled, deionized water. This stock will be stored tightly sealed and protected from light. A 220 mM stock solution of HCl will be prepared by dilution of concentrated acid (Fisher #A144-212) and deoxygenated by dissolving with compressed nitrogen.

### Steps

1. Oxygen-free distilled, deionized water will be added to a three neck flask and deoxygenated by dissolving with nitrogen for 30 minutes while stirring.
2. 2.5 M Na₂S Stock will be added to yield a 200 mM Na₂S solution.
3. The 200 mM Na₂S Solution will be bubbled with compressed nitrogen for 15 minutes while stirring.
4. Nitric oxide gas will be bubbled into the Na₂S solution in an oxygen free environment.
   pH will be adjusted to a final pH of 7.0-8.0 while dissolving with compressed nitrogen and stirring.

### Composition 2: Nitric Oxide Liquid and Hydrogen Sulfide Liquid

### Starting Materials

Nitric oxide liquid composition: In one embodiment, aqueous nitric oxide will be prepared by saturating pure NO gas and hydrolyzing 1 mM 1-hydroxy-2-oxo-3(N-methyl-3-aminoethyl)-3-methyl-1-triazene (NOC-7), in an oxygen-free environment using a modified Saltzman method (see: Ohkawa et al., Nitric Oxide, (2001) 5:515).

Liquid composition: Stock solutions will be prepared using deoxygenated water. The water will be deoxygenated by removing air under vacuum and dissolving with compressed nitrogen (99.99%) for 30 minutes. A saturated stock solution of 2.5 M Na₂S will be prepared from Na₂S.9H2O crystals (Fisher #5425) that will be rinsed with oxygen-free, distilled, deionized water. This stock will be stored tightly sealed and protected from light. A 220 mM stock solution of HCl will be prepared by dilution of concentrated acid (Fisher #A144-212) and deoxygenated by dissolving with compressed nitrogen.

### Steps

1. Oxygen-free distilled, deionized water will be added to a three neck flask and deoxygenated by dissolving with nitrogen for 30 minutes while stirring.
2. 2.5 M Na₂S Stock will be added to yield a 200 mM Na₂S solution.
3. The 200 mM Na₂S Solution will be bubbled with compressed nitrogen for 15 minutes while stirring.
4. Nitric oxide liquid (prepared as described in the foregoing) will be combined with Na₂S solution.
   pH will be adjusted to a final pH of 7.0-8.0 while dissolving with compressed nitrogen and stirring.

Any order may be used to add Na₂S and nitric oxide liquid together.

### Composition 3: Nitric Oxide Liquid and Hydrogen Sulfide Gas

Nitric oxide liquid composition: In one embodiment, aqueous nitric oxide will be prepared by saturating pure NO gas and hydrolyzing 1 mM 1-hydroxy-2-oxo-3(N-methyl-3-aminoethyl)-3-methyl-1-triazene (NOC-7), in an oxygen-free environment using a modified Saltzman method (see: Ohkawa et al., Nitric Oxide, (2001) 5:515).

### Steps

1. Oxygen-free distilled, deionized water will be added to a three neck flask and deoxygenated by dissolving with nitrogen for 30 minutes while stirring.
2. 2.5 M Na₂S Stock will be added to yield a 200 mM Na₂S solution.
3. The 200 mM Na₂S Solution will be bubbled with compressed nitrogen for 15 minutes while stirring.
4. Hydrogen sulfide gas will be bubbled into the nitric oxide solution in an oxygen-free environment.
   pH will be adjusted to a final pH of 7.0-8.0 while dissolving with compressed nitrogen and stirring.

### Example 4

### Methods of Manufacturing Liquid Sulfide Compositions

Four liquid pharmaceutical sulfide compositions were prepared as described below.

Stock solutions were prepared using deoxygenated water. The water was deoxygenated by removing air under vacuum and dissolving with compressed nitrogen (99.99%) for 30 minutes. A saturated stock solution of 2.5 M Na₂S was prepared from Na₂S.9H2O crystals (Fisher #5425) that were rinsed with oxygen-free, distilled, deionized water. This stock was stored tightly sealed and protected from light. A 220 mM stock solution of HCl was prepared by dilution of concentrated acid (Fisher #A144-212) and deoxygenated by dissolving with compressed nitrogen.

Liquid pharmaceutical compositions were prepared in a fume hood in a basic glove box filled with nitrogen gas to yield an oxygen-free environment. The reactor with pH meter, bubbler and stirrer were in the glove box. Oxygen levels in the glove box were monitored with an oxygen meter (Mettler-Toledo) with a sensitivity level of 0.03 μM. Methods of preparing the liquid pharmaceutical compositions of the present invention include limiting oxygen content in each aspect of manufacturing and storage of the pharmaceutical composition where oxygen is measured in the range of 0 μM-5 μM in the pharmaceutical composition.

Liquid pharmaceutical compositions were prepared in a three-neck flask (Wilmad Labs) with each opening fitted with ground glass fittings having the following features:
d) A universal adapter with a plastic cap with a central orifice and o-ring. This adapter was fitted with a pH probe and sealed by the O-ring.
e) Universal adapter with a hose connector and a plastic cap with a central orifice and O-ring. This adapter was fitted with a gas dispersion tube with a glass frit. The dispersion tube was connected to a compressed gas cylinder and used to deoxygenate the solution by dissolving with compressed nitrogen and to neutralize the pH with a mixture of H₂S and nitrogen. The hose connector was fitted with a plastic tube to allow pressure to escape. These two connections were reversed to dispense the contents of the flask under positive nitrogen pressure.
f) The third neck was sealed with a ground glass stopper and used to add Na₂S solution or water to the flask.
   1. Liquid Pharmaceutical Composition I—Na₂S nonahydrate

Liquid Pharmaceutical Composition I was prepared with the following steps:
a) Oxygen-free distilled, deionized water was added to a three neck flask and deoxygenated by dissolving with nitrogen for 30 minutes while stirring.
b) 2.5 M Na₂S Stock was added to yield a 200 mM Na₂S solution.
c) The 200 mM Na₂S Solution was bubbled with compressed nitrogen for 15 minutes while stirring.
d) 220 mM HCl was added until a final pH of 7.8-8.0 while dissolving with compressed nitrogen and stirring.
e) Deoxygenated deioinized water was added to give a final concentration of 100 mM Na₂S.
   2. Liquid Pharmaceutical Composition II—Na₂S nonahydrate

Liquid Pharmaceutical Composition II was prepared with the following steps:
a) Deionized, oxygen-free water was added to the three neck flask and deoxygenated by dissolving with nitrogen for 30 minutes while stirring.
b) 2.5 M Na₂S Stock was added to yield a 100 mM Na₂S solution.
c) The 100 mM Na₂S Solution was bubbled with compressed nitrogen for 15 minutes while stirring.
d) The solution was bubbled with a 50/50 mixture of compressed nitrogen and CO₂ (99.9%) until a pH of 7.8 was reached.
   3. Liquid Pharmaceutical Composition III—Na₂S with H₂S and Nitrogen

Liquid Pharmaceutical Composition III was prepared with the following steps:
a) Deionized, oxygen-free water was added to the three neck flask and deoxygenated by dissolving with nitrogen for 30 minutes while stirring.
b) 2.5 M Na₂S Stock was added to yield a 100 mM Na₂S solution.
c) The 100 mM Na₂S Solution was bubbled with compressed nitrogen for 15 minutes while stirring.
d) The solution was bubbled with a 50/50 mixture of compressed nitrogen and H₂S until a pH of 8.2 was reached. This resulted in a final concentration of 90 mM sulfide.

### 4. Liquid Pharmaceutical Composition IV-H₂S

The final sulfide concentration of Liquid Pharmaceutical Composition IV was determined by the initial concentration of NaOH. Liquid Pharmaceutical Composition IV was prepared with the following steps:
a) NaOH in a range of 5 mM to 500 mM solution was added to the three neck flask with additives (DTPA, anti-oxidants) (FIG. 1.)
b) The solution was deoxygenated by bubbling with argon at 5 psi for 15 minutes while stirring.
c) H₂S was bubbled through the solution while stirring until pH was reduced to 7.7 (or a range of 7.6 to 7.8).
d) The headspace in the flask was flushed with argon.
e) Amber dispensing bottles or vials were placed in a glove box that was flushed with a constant stream of argon and each bottle or vial was flushed with argon.
f) The formulation was dispensed under argon to maintain an oxygen-free environment.

The stability of the solution was monitored by measurement of sulfide concentration, pH, and absorbance spectrum (polysulfide formation). Additional assays were performed to monitor oxidation products which include sulfite, sulfate, thiosulfate, and elemental sulfur.

Liquid pharmaceutical compositions were dispensed within the sealed Glove box, from the three-necked flask under positive nitrogen pressure. Amber vials or amber bottles were filled to a slight over-pressure in an inert atmosphere argon or nitrogen to prevent/slow oxidative breakdown of the liquid pharmaceutical compositions, and sealed with plastic caps with Teflon/silicon liners or plastic caps with central Teflon lined silicon septa using a crown-cap crimper (Aldrich Z112976) to provide an air-tight seal.

### Example 5

### Methods of Manufacturing No in a Pharmaceutically Acceptable Buffer

Two methods for preparing an aqueous formulation of NO are described (see, Ohkawa et al., Nitric Oxide, (2001) 5:515).

According to one method, a 100-ml NO solution in 0.1M phosphate buffer (pH 7.4) was prepared using pure NO gas. NO₂ contamination was minimized. NO gas was purified by a column with a KOH pellet to remove NO₂ in the NO gas tank generated by the dismutation reaction: 3NO→NO₂+N₂O before introduction into the buffer. A column of sodium hydrosulfite on glass wool was attached to avoid exposure of the flask content to atmospheric oxygen. Nitrogen gas was purged to remove NO in the headspace of the flask to avoid conversion of gaseous NO into NO₂ in contact with atmospheric oxygen.

The following five steps were then followed: (1) 0.1 M phosphate buffer (pH 7.4) (100 ml) was placed in the flask and the flask was tightly sealed with a silicone stopper; (2) the solution was kept at 20° C. and gently stirred; (3) nitrogen gas was introduced through the cock at 70 ml/min for 3 h; (4) NO gas was introduced through the cock at 10 ml/min for 17 min; and (5) for determination of the nitrogen oxide species in the aqueous solution, 1.0 ml of the solution was withdrawn by means of a gas-tight syringe through a silicone stopper. For determination of the nitrogen oxide species in the aqueous solution generated in contact with oxygen, the silicone stopper was removed from the flask and 1.0 ml of the solution was withdrawn after keeping the solution at 20° C. for the indicated period under the aerobic conditions.

A second method of manufacture used NOC-7, which releases 2 equivalent amounts of NO in a neutral solution. A 100-ml NO solution in 0.1 M phosphate buffer (pH 7.4) was prepared from NOC-7. The first three steps were followed the same as described in the foregoing, except that the volume of the phosphate buffer was 90 ml, and the temperature of the flask was maintained at 37° C. During a fourth step, a 10-ml solution of 10 mM NOC-7 in 0.1 M NaOH, which had been deoxygenated by purging nitrogen gas, was introduced by means of a gas-tight syringe through the silicon stopper, and the mixture was maintained at 37° C. for 1 h, after which the temperature of the mixture was made at 20° C. Step 5 was the same as described in the foregoing. All of the above U.S. patents, U.S. patent application publications,

U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of reducing a cytotoxic effect of nitric oxide in a biological matter, comprising administering to the biological matter nitric oxide in combination with sulfide.

2. A method of reducing a cytotoxic effect of sulfide in a biological matter, comprising administering to the biological matter sulfide in combination with nitric oxide.

3-4. (canceled)

5. The method of claim 1 or claim 2, wherein said nitric oxide is administered as a gas and said sulfide is administered as a liquid.

6. (canceled)

7. The method of claim 1 or 2, wherein said nitric oxide and said sulfide are administered concurrently.

8. The method of claim 1, wherein said sulfide is administered prior to administration of said nitric oxide.

9. The method of claim 2, wherein said nitric oxide is administered prior to administration of said sulfide.

10. A method of treating or preventing a respiratory, cardiovascular, pulmonary, or blood disease or disorder, a tumor, an infection, inflammation, shock, sepsis, or stroke, in a patient, comprising administering to a patient an effective amount of nitric oxide in combination with sulfide.

11. A method of preventing or reducing injury to, or enhancing survivability of, a biological material exposed to ischemic or hypoxic conditions, comprising contacting the biological material with an effective amount of sulfide in combination with nitric oxide.

12-14. (canceled)

15. The method of claim 11, wherein the ischemic or hypoxic conditions result from an injury to the biological material, the onset or progression of a disease that adversely affects the biological material, or hemorrhaging of the biological material, and wherein the biological material is contacted with sulfide and nitric oxide before the injury, before the onset or progression of the disease, or before hemorrhaging of the biological material.

16-17. (canceled)

18. The method of claim 11, wherein the biological material is selected from the group consisting of cells, tissues, organs, organisms, and mammals.

19-27. (canceled)

28. A device for the metered coadministration of nitric oxide and sulfide to a patient, characterized by a gas feed system including a first line feeding nitric oxide, a second line feeding sulfide, a shut-off valve in the first line, a shut-off valve in the second line, wherein the first and second lines are in flow communication with a third line, whereby upon opening both shut-off valves to open flow nitric oxide and sulfide may flow through the first and second lines and into the third line, where they are mixed, and a device for delivering the resulting mixture of nitric oxide and sulfide to the patient, wherein said device is in flow communication with the third line.

29. The device of claim 28, further comprising a fourth line feeding air and a shut-off valve in the fourth line, wherein the fourth line is in flow communication with the third line, whereby upon opening all shut-off valves to open flow nitric oxide, sulfide, and air may flow through the first, second, and third lines and into the third line, where they are mixed.

30-32. (canceled)

33. The method of any one of claim 1, 2, 10, or 11, wherein the administering or contacting is performed intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, or via a lavage.

34-73. (canceled)

74. A method of preparing a composition of nitric oxide and sulfide suitable for administration to an animal, comprising:
a) saturating nitric oxide gas in a liquid;
b) dissolving an aqueous solution of deoxygenated water and HS⁻, wherein the aqueous solution has an oxygen content less than or equal to 5 μM, into the composition resulting from step (a); and
c) adjusting the pH of the composition resulting from step (b) to a pH in the range of 6.5 to 8.5, 7.3 to 7.6, 5.0 to 9.0, 6.0 to 8.5, or 7.0 to 8.0 by adding hydrogen chloride or sodium hydroxide, thereby producing a liquid composition of nitric oxide and sulfide suitable for administration to an animal.

75-81. (canceled)

82. The method of claim 74, further comprising adjusting the osmolarity of the composition resulting from step (c) to an osmolarity in the range of 250-350 mOsmol/L.

83-85. (canceled)

86. The method of claim 74, wherein the oxygen content of the composition is less than or equal to 5 μM for about six months.

87-88. (canceled)

89. The method of claim 74, wherein said HS⁻is made by dissolving in water a compound selected from the group consisting of: H₂S, Na₂S, NaHS, K₂S, KHS. Rb₂S, CS₂S, (NH₄)₂S, (NH₄)HS, BeS, MgS, CaS, SrS, and BaS and hydrates thereof.

90. The method of claim 11, wherein the ischemic or hypoxic conditions results from stroke or coronary artery bypass graft (CABG) surgery.
